(19) United States
(12) Patent Application Publication
(10) Pub. No.: US 2025/0384966 A1
(43) Pub. Date: Dec. 18, 2025

(54) Systems and Methods for Automated Compound Synthesis

(71) Applicant: DeepCure Inc., Boston, MA (US)

(72) Inventors: Heather Osswald, San Diego, CA (US); Luke Nam, Cambridge, MA (US); Derrick Miyao, San Diego, CA (US); Thrasyvoulos Karydis, Boston, MA (US)

(21) Appl. No.: 19/229,972

(22) Filed: Jun. 5, 2025

Related U.S. Application Data
(60) Provisional application No. 63/660,389, filed on Jun. 14, 2024.

(51) Int. Cl. G16C 20/10; G16C 20/70

(52) U.S. Cl. CPC G16C 20/10; G16C 20/70

(57) ABSTRACT

An apparatus for improving a model for use in optimizing a multistep molecular reaction is provided. The apparatus includes an automated synthesis platform, and a computing system comprising one or more processors and memory addressable by the one or more processors. A plurality of instances of the molecular reaction is performed using synthons and normalized conditions. For each respective instance, at least a subset of the synthons is transformed using the molecular reaction, generating compounds. For each respective instance, a respective conversion value is obtained. A subset of instances is selected based on at least a threshold conversion value for the respective conversion value of each respective instance. The subset of instances is used to adjust one or more parameters in a plurality of parameters of the model, obtaining an updated plurality of parameters for the model. Using, subsequent to obtaining the updated plurality of parameters, the model to search for and identify an updated plurality of normalized conditions for the molecular reaction that collectively have an improved conversion value for the molecular reaction relative to the original plurality of normalized conditions.

### CROSS REFERENCE TO RELATED APPLICATIONS

This application claims priority to U.S. Provisional Patent Application No. 63/660,389 entitled “Systems and Methods for Automated Compound Synthesis,” filed Jun. 14, 2024, which is hereby incorporated by reference.

### TECHNICAL FIELD

This application is directed to apparatuses and methods for generating compounds from synthons, in particular using optimized molecular reaction conditions obtained from models.

### BACKGROUND

Pharmaceutical companies spend millions of dollars screening compounds to discover novel compounds and develop them into prospective drug leads. Traditionally, this has involved collecting and testing large libraries of compounds to find a small number of compounds that interact with the disease target of interest. Unfortunately, the cost and time needed to physically assay compounds is prohibitive to testing them at scale.

Despite decades of effort and millions of dollars spent on end-to-end automation, drug discovery is conventionally driven by manual lab processes. End-to-end automated platforms have largely fallen short of expectations because traditional automation relies on worklists designed around single, fixed-input processes. These traditional worklists are unsuitable for driving complex, multi-instrument workflows with dynamically changing parameters. Further, traditional worklists require manual customization for each iteration of the design-make-test cycle.

Given the above background, what is needed in the art are improved methods for designing, developing, and/or synthesizing compounds for drug discovery.

### SUMMARY

The present disclosure addresses the problems identified in the background by providing systems and methods that make use of machine learning models to facilitate development, synthesis, and/or screening of compounds for drug discovery. In particular, the disclosed apparatuses and systems utilize a framework for dynamic generation of molecular reaction conditions to enable automation of such processes. Advantageously, in some implementations, the disclosed apparatuses and systems allow for compound development, synthesis, and screening within a single platform. Moreover, in some implementations, the disclosed apparatuses and systems are agnostic to the type of automated workflow used and removes the need for scientists to review outputs between stages of execution. In some implementations, the disclosed apparatuses and systems also enable different software to communicate directly and exchange information so that generated worklists containing molecular reaction conditions can be automatically re-configured for subsequent cycles of development, synthesis, and/or screening. This framework provides a foundation for improved end-to-end automated chemical synthesis and compound testing for drug discovery using machine learning models.

Accordingly, one aspect of the present disclosure provides an apparatus for improving a first model (e.g., a reinforcement learning model) for use in optimizing a molecular reaction, where the molecular reaction is a multistep molecular reaction. In some embodiments, the apparatus includes an automated synthesis platform, and a computing system comprising one or more processors and memory addressable by the one or more processors, the memory storing the first model. In some embodiments, the optimization includes selecting the first molecular reaction using the computing system, wherein the computing system informs the automated synthesis platform of the first molecular reaction. In some embodiments, the optimization further includes performing a first plurality of instances of the first molecular reaction using a first plurality of at least 4 synthons and an original plurality of normalized conditions using the automated synthesis platform. For each respective instance of the first molecular reaction, a transformation, with the automated synthesis platform, of at least a subset of the first plurality of synthons occurs using the first molecular reaction, thereby generating a first plurality of compounds. In some embodiments, the optimization further includes obtaining, for each respective instance of the first molecular reaction, a respective conversion value for the respective instance. A first subset of instances is selected from the first plurality of instances based on at least a first threshold conversion value for the respective conversion value of each respective instance. The automated synthesis platform informs the computing system of the first subset of instances.

In some embodiments, the optimization further includes training the first model by using i) the first subset of instances as independent variables and ii) the corresponding conversion value of each instance of the first subset of instances as dependent variables, to guide adjustment of one or more parameters in a plurality of parameters of the first model, so that the first model produces a calculated conversion value in agreement with the corresponding conversion value of each instance of the first subset of instances upon input of the first subset of instances into the first model. In some embodiments, the optimization further includes using, subsequent to obtaining the updated plurality of parameters, the first model to search for and identify an updated plurality of normalized conditions for the first molecular reaction that collectively have an improved conversion value for the first molecular reaction relative to the original plurality of normalized conditions.

In some embodiments, the first model is a graph neural network. In some embodiments, the graph neural network is pre-trained, prior to the training, on a local level using a plurality of unlabeled molecules. In some embodiments, the plurality of unlabeled molecules is other than the plurality of compounds. In some embodiments, the plurality of unlabeled molecules is the ZINC15 database.

In some embodiments, each respective instance in the first subset of instances is a corresponding graph comprising a corresponding plurality of nodes and a corresponding plurality of edges. Each respective node in the corresponding plurality of nodes is a synthon used in the respective instance, and each edge in the corresponding plurality of edges is between a first node and a second node in the corresponding plurality of nodes and is associated with at least a conversion efficiency in the respective instance between the first node and the second node.

In some embodiments, the search for and identification of an updated plurality of normalized conditions for the first molecular reaction that collectively have an improved conversion value for the first molecular reaction relative to the original plurality of normalized conditions is performed in accordance with a reinforcement learning policy in which the first model is used as an oracle for the reinforcement learning policy.

In some embodiments, an amount of each respective synthon in the first plurality of synthons used in each respective instance of the first molecular reaction is in a first reaction amount range.

In some embodiments, the first reaction amount range is 0.0005 millimoles and 0.005 millimoles or 0.002 millimoles and 1.5 millimoles of the respective synthon. In some embodiments, the first reaction amount range is between 150 g/mol and 300 g/mol of the respective synthon. In some embodiments, an absolute volume of each instance of the first molecular reaction in the plurality of instances of the first molecular reaction is in a first reaction volume range. In some embodiments, the first reaction volume range is between 10 microliters and 1800 microliters. In some embodiments, each edge in the corresponding plurality of edges is further associated with any combination of a solvent, a concentration, a temperature, a reaction volume, an incubation time, a stoichiometry of synthons, or a stoichiometry of reagents.

In some embodiments, the optimization further includes performing a second plurality of instances of the first molecular reaction using (a) the first plurality of synthons and the updated plurality of normalized conditions and (b) the automated synthesis platform. In some embodiments, for each respective instance of the first molecular reaction, a transformation, with the automated synthesis platform, of at least a subset of the plurality of synthons occurs using the first molecular reaction, thereby generating a second plurality of compounds. In some embodiments, the optimization further includes obtaining, for each respective instance of the first molecular reaction, a respective conversion value for the respective instance. In some embodiments, the optimization further includes selecting a second subset of instances from the plurality of instances based on at least the first threshold conversion value for the respective conversion value of each respective instance. In some such embodiments the automated synthesis platform informs the computing system of the subset of instances. In some embodiments, the optimization further includes retraining the first model by using i) the second subset of instances as independent variables and ii) the corresponding conversion value of each instance of the second subset of instances as dependent variables, to guide adjustment of one or more parameters in the plurality of parameters of the first model, so that the first model produces a calculated conversion value in agreement with the corresponding conversion value of each instance of the second subset of instances upon input of the subset of instances into the first model. In some embodiments, the optimization further includes using, subsequent to obtaining the updated plurality of parameters, the first model to search for and identify a reupdated plurality of normalized conditions for the first molecular reaction that collectively has an improved conversion value for the first molecular reaction relative to the updated plurality of normalized conditions.

In some embodiments, the model training is in accordance with a loss function, an assent function, or a regression. In some embodiments, the plurality of unlabeled molecules comprises 1000 or more unlabeled molecules or 10,000 or more unlabeled molecules. In some embodiments, the plurality of unlabeled molecules comprises 1×10⁶ or more unlabeled molecules. In some embodiments, each compound in the first plurality of compounds is an organic compound having a molecular weight of less than 500 Daltons, less than 1000 Daltons, less than 2000 Daltons, less than 4000 Daltons, less than 6000 Daltons, less than 8000 Daltons, less than 10000 Daltons, or less than 20000 Daltons. In some embodiments, each compound in the first plurality of compounds is an organic compound having a molecular weight of between 400 Daltons and 10000 Daltons. In some embodiments, the first plurality of compounds comprises 100 or more, 500 or more, 1000 or more, 2000 or more or 10,000 or more compounds.

In some embodiments, each compound in the first plurality of compounds satisfies two or more rules, three or more rules, or all four rules of the Lipinski's rule of Five: (i) not more than five hydrogen bond donors, (ii) not more than ten hydrogen bond acceptors, (iii) a molecular weight under 500 Daltons, and (iv) a Log P under 5. In some embodiments, the plurality of parameters comprises 500,000 or more parameters, or 1×10⁶ or more parameters.

In some embodiments, the first model is a deep neural network, and the first model further generates, as output, an uncertainty estimation for the improved conversion value for the first molecular reaction.

In some embodiments, the first plurality of compounds includes one or more first reaction intermediates.

In some embodiments, the optimization further includes selecting a second molecular reaction using the computing system. The second molecular reaction is in the multistep synthesis. The computing system informs the automated synthesis platform of the second molecular reaction. In some embodiments, the optimization further includes selecting a subset of the first plurality of compounds based on at least the first threshold conversion value of each respective compound of the first plurality of compounds. In some embodiments, the optimization further includes converting each respective compound of the subset of the first plurality of compounds into a corresponding synthon to provide a second plurality of synthons. In some embodiments, the optimization further includes performing a second plurality of instances of the second molecular reaction using the second plurality of synthons and a second original plurality of normalized conditions using the automated synthesis platform. In some embodiments, for each respective instance of the second molecular reaction, a transformation, with the automated synthesis platform, of at least a subset of the second plurality of synthons occurs using the second molecular reaction, thereby generating a second plurality of compounds. In some embodiments, the optimization further includes obtaining, for each respective instance of the second molecular reaction, a second respective conversion value for the respective instance. In some embodiments, the optimization further includes selecting a second subset of instances from the second plurality of instances based on at least a second threshold conversion value for the second respective conversion value of each respective instance, where the automated synthesis platform informs the computing system of the second subset of instances. In some embodiments, the optimization further includes training a second model by using i) the second subset of instances as independent variables and ii) the corresponding conversion value of each instance of the second subset of instances as dependent variables, to guide adjustment of one or more parameters in a plurality of parameters of the second model, so that the second model produces a calculated conversion value in agreement with the corresponding conversion value of each instance of the second subset of instances upon input of the second subset of instances into the second model.

In some embodiments, the first model and the second model are the same model. In some embodiments, the first model and the second model are different models. In some embodiments, the transforming further includes reacting each respective synthon of the second subset of the second plurality of synthons with a synthon of a third plurality of synthons.

In some embodiments, each respective synthon of the third plurality of synthons is prepared by converting a compound prepared using the apparatus of the disclosure into the respective synthon.

In some embodiments, the first plurality of synthons comprises at least 1×10⁶ initial synthons.

In some embodiments, each respective normalized condition in the first plurality of normalized conditions and/or the second plurality of normalized conditions is selected from the group consisting of: synthon type, reagents, solvents, concentrations, order of addition, amount of equivalents for addition, synthon scope, temperature, incubation time, stoichiometry of synthons, and stoichiometry of reagents. In some embodiments, the first plurality of instances and/or the second plurality of instances of the molecular reaction comprises at least 1×10⁶ instances.

In some embodiments, the respective conversion value is a percent yield of a corresponding compound obtained for the respective instance of the first molecular reaction determined as a ratio of product to starting material, in which the first threshold conversion value is at least 20%. In some embodiments, the second respective conversion value is a percent yield of a corresponding compound obtained for the respective instance of the second molecular reaction determined as a ratio of product to starting material, in which the second threshold conversion value is at least 20%. In some embodiments, the first threshold conversion value is at least 40%, at least 50%, or at least 60%. In some embodiments, the first threshold conversion value is at least 40%, at least 50%, or at least 60%.

In some embodiments, the first molecular reaction and/or the second molecular reaction comprises at least 2, at least 3, or at least 4 steps. In some embodiments, the first molecular reaction comprises at least 2, at least 3, or at least 4 steps.

Another aspect of the present disclosure includes an apparatus for automating synthesis of a compound using a first molecular reaction. In some embodiments, the first molecular reaction is a multistep synthesis. In some embodiments, the apparatus includes an automated synthesis platform, and a computing system comprising one or more processors and memory addressable by the one or more processors, the memory storing a first model. In some embodiments, the automating includes selecting the first molecular reaction. In some embodiments, the automating further includes performing a first plurality of instances of the first molecular reaction using a first plurality of at least 4 synthons and a plurality of normalized conditions using the automated synthesis platform. For each respective instance of the first molecular reaction, a transformation, with the automated synthesis platform, of at least a subset of the first plurality of synthons occurs using the first molecular reaction, thereby generating a first plurality of compounds. In some embodiments, the automating further includes obtaining, for each respective instance of the first molecular reaction, a respective conversion value for the respective instance. In some embodiments, the automating further includes selecting a first subset of instances from the first plurality of instances based on at least a first threshold conversion value for the respective conversion value of each respective instance, where the automated synthesis platform informs the computing system of the first subset of instances. In some embodiments, the automating further includes training the first model by using i) the first subset of instances as independent variables and ii) the corresponding conversion value of each instance of the first subset of instances as dependent variables, to guide adjustment of one or more parameters in a plurality of parameters of the first model, so that the first model produces a calculated conversion value in agreement with the corresponding conversion value of each instance of the first subset of instances upon input of the first subset of instances into the first model.

Another aspect of the present disclosure includes an apparatus for identifying synthons for a molecular reaction. In some embodiments, the molecular reaction is a multistep synthesis. In some embodiments, the apparatus includes an automated synthesis platform, and a computing system comprising one or more processors and memory addressable by the one or more processors. In some embodiments, the identifying includes selecting the molecular reaction. In some embodiments, the identifying further includes performing a first plurality of instances of the molecular reaction using a plurality of at least 4 synthons and a plurality of normalized conditions using the automated synthesis platform. For each respective instance of the molecular reaction, a transformation, with the automated synthesis platform, of at least a subset of the first plurality of synthons occurs using the molecular reaction, thereby generating a plurality of compounds. In some embodiments, the identifying further includes obtaining, for each respective instance of the molecular reaction, a respective conversion value for the respective instance. In some embodiments, the identifying further includes selecting a subset of instances from the plurality of instances based on at least a threshold conversion value for the respective conversion value of each respective instance. In some embodiments, the identifying further includes selecting a subset of synthons that are enriched in the subset of instances relative to the plurality of instances of the molecular reaction, thereby identifying synthons for the molecular reaction.

### BRIEF DESCRIPTION OF THE DRAWINGS

In the drawings, embodiments of the systems and methods of the present disclosure are illustrated by way of example. It is to be expressly understood that the description and drawings are only for the purpose of illustration and as an aid to understanding, and are not intended as a definition of the limits of the systems and methods of the present disclosure.

FIG. 1 collectively illustrates an apparatus in accordance with some embodiments of the present disclosure.

FIGS. 2A-2C collectively illustrate example workflow for improving a model (e.g., a reinforcement learning model) for use in optimizing a molecular reaction (e.g., a multistep molecular reaction), in which optional steps are indicated by dashed lines, in accordance with some embodiments of the present disclosure.

FIG. 3 illustrates an automated synthesis platform in accordance with some embodiments of the present disclosure.

FIGS. 4A and 4B collectively illustrate a comparison of predicted properties for candidate molecules obtained using machine learning approaches compared to candidate molecules obtained from a reference compound library, in accordance with an embodiment of the present disclosure. FIG. 4A illustrates example predictions of target inhibition. FIG. 4B illustrates example predictions of absorption, distribution, metabolism, and excretion (ADME) scores.

Like reference numerals refer to corresponding parts throughout the several views of the drawings.

### DETAILED DESCRIPTION

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

The present disclosure addresses the problems identified in the background by providing systems and methods that make use of machine learning models to facilitate development, synthesis, and/or screening of compounds for drug discovery. In particular, the disclosed systems and methods utilize a framework for dynamic generation of molecular reaction conditions to enable automation of such processes.

Combining automation, chemistry, and machine learning can overcome human limitations in drug discovery. For instance, manual chemistry often leads to performing more of what an individual already knows. Typically, chemists approach drug design one parameter at a time, in addition to designing and synthesizing compounds one at a time. As such, the limitations of manual chemistry can impede the design of new molecules. Conversely, an automated chemical synthesis platform is as powerful as the reactions it can perform. More reactions equals more chemical space, which in turn enables machine learning tools to design and access a greater scope of multiparameter-designed molecules. Utilizing recent increases in computational power, an automated synthesis platform connected to compound screening and testing can enable standardized big data that have never before been possible. Such data can lead to improved models and designs of new molecules for drug discovery.

Advantageously, in some implementations, the disclosed systems and methods allow for compound development, synthesis, and screening within a single platform (e.g., “design-make-test”). Moreover, in some implementations, the disclosed systems and methods are agnostic to the type of automated workflow used and remove the need for scientists to review outputs between stages of execution. In some implementations, the disclosed systems and methods also enable different software to communicate directly and exchange information so that generated worklists containing molecular reaction conditions can be automatically re-configured for subsequent cycles of development, synthesis, and/or screening. This framework provides a foundation for improved end-to-end automated chemical synthesis and compound testing for drug discovery using machine learning models.

Accordingly, the present disclosure provides systems and methods for improving a model (e.g., a reinforcement learning model) for use in optimizing a molecular reaction (e.g., a multistep molecular reaction). A plurality of instances of the molecular reaction is performed using synthons and normalized conditions. For each respective instance, at least a subset of the synthons is transformed using the molecular reaction. A plurality of compounds is thereby generated. For each respective instance, a respective conversion value is also obtained. A subset of instances is selected from the plurality of instances based on at least a threshold conversion value for the respective conversion value of each respective instance. For instance, in some embodiments, the respective conversion value of each respective instance is compared to the threshold conversion value for selection of the subset of instances. The subset of instances is used to adjust one or more parameters in a plurality of parameters of the model, obtaining an updated plurality of parameters for the model. Subsequent to updating the plurality of parameters, responsive to inputting the plurality of synthons into the model with the updated plurality of parameters, an updated plurality of normalized conditions for the molecular reaction is produced as output from the model.

It will be understood that, although the terms first, second, etc. may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject.

### Definitions

The terminology used in the present disclosure is for the purpose of describing particular embodiments only and is not intended to be limiting of the invention. As used in the description of the invention and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

As used interchangeably herein, the terms “macromolecule,” “macromolecule complex,” or “polymer” refer to a biological object that is capable of interacting with a molecule. In some embodiments, a macromolecule is a protein, a polypeptide, a polynucleic acid, a polyribonucleic acid, a polysaccharide, or an assembly of any combination thereof. In some embodiments, a macromolecule is a large molecule composed of repeating residues. In some embodiments, the macromolecule is a natural material. In some embodiments, the macromolecule is a synthetic material. In some embodiments, the macromolecule is an elastomer, shellac, amber, natural or synthetic rubber, cellulose, Bakelite, nylon, polystyrene, polyethylene, polypropylene, polyacrylonitrile, polyethylene glycol, or a polysaccharide. In some embodiments, the macromolecule is a heteropolymer (copolymer). In some embodiments, the macromolecule is a plurality of polymers (e.g., 2 or more, 3, or more, 10 or more, 100 or more, 1000 or more, or 5000 or more polymers), where the respective polymers in the plurality of polymers do not all have the same molecular weight. In some embodiments, the macromolecule is a polypeptide. As used herein, the term “polypeptide” means two or more amino acids or residues linked by a peptide bond.

In some embodiments, the macromolecule includes any number of posttranslational modifications. Thus, in some embodiments, a macromolecule includes those polymers that are modified by acylation, alkylation, amidation, biotinylation, formylation, γ-carboxylation, glutamylation, glycosylation, glycylation, hydroxylation, iodination, isoprenylation, lipoylation, cofactor addition (for example, of a heme, flavin, metal, etc.), addition of nucleosides and their derivatives, oxidation, reduction, pegylation, phosphatidylinositol addition, phosphopantetheinylation, phosphorylation, pyroglutamate formation, racemization, addition of amino acids by tRNA (for example, arginylation), sulfation, selenoylation, ISGylation, SUMOylation, ubiquitination, chemical modifications (for example, citrullination and deamidation), and treatment with other enzymes (for example, proteases, phosphatases and kinases). Other types of posttranslational modifications are known in the art and are within the scope of the macromolecules or macromolecule complexes of the present disclosure.

In some embodiments, the macromolecule is a surfactant. In some embodiments, the macromolecule is a reverse micelle or liposome. In some embodiments, the target macromolecule is a fullerene. In some embodiments, the macromolecule includes two different types of polymers, such as a nucleic acid bound to a polypeptide. In some embodiments, the target macromolecule includes two polypeptides bound to each other. In some embodiments, the target macromolecule includes one or more metal ions (e.g., a metalloproteinase with one or more zinc atoms).

As used herein, the term “target” refers to an object of interest, such as a macromolecule, macromolecule complex, or polymer that is of interest as a primary binding target for a candidate molecule. As used herein, the term “off-target” refers to an object that is not the primary binding target, such as a macromolecule, macromolecule complex, or polymer that exhibits off-target binding with a candidate molecule.

As used herein, the terms “model”, “regressor”, and/or “classifier” interchangeably refer to a machine learning model.

In some embodiments, a model is a supervised machine learning model. Nonlimiting examples of supervised learning models include, but are not limited to, logistic regression, neural networks, support vector machines, Naive Bayes models, nearest neighbor models, random forest models, decision tree models, boosted trees models, multinomial logistic regression, linear models, linear regression, GradientBoosting, mixture models, hidden Markov models, Gaussian NB models, linear discriminant analysis, or any combinations thereof.

Neural networks. In some embodiments, the model is a neural network (e.g., a convolutional neural network and/or a residual neural network). Neural networks, also known as artificial neural networks (ANNs), include convolutional and/or residual neural network models (deep learning algorithms). Neural networks can be machine learning models that may be trained to map an input data set to an output data set, where the neural network comprises an interconnected group of nodes organized into multiple layers of nodes. For example, the neural network architecture may comprise at least an input layer, one or more hidden layers, and an output layer. The neural network may comprise any total number of layers, and any number of hidden layers, where the hidden layers function as trainable feature extractors that allow mapping of a set of input data to an output value or set of output values. As used herein, a deep learning model can be a neural network comprising a plurality of hidden layers, e.g., two or more hidden layers. Each layer of the neural network can comprise a number of nodes (or “neurons”). A node can receive input that comes either directly from the input data or the output of nodes in previous layers, and perform a specific operation, e.g., a summation operation. In some embodiments, a connection from an input to a node is associated with a parameter (e.g., a weight and/or weighting factor). In some embodiments, the node may sum up the products of all pairs of inputs, Xi, and their associated parameters. In some embodiments, the weighted sum is offset with a bias, b. In some embodiments, the output of a node or neuron may be gated using a threshold or activation function, f, which may be a linear or non-linear function. The activation function may be, for example, a rectified linear unit (ReLU) activation function, a Leaky ReLU activation function, or other function such as a saturating hyperbolic tangent, identity, binary step, logistic, arcTan, softsign, parametric rectified linear unit, exponential linear unit, softPlus, bent identity, softExponential, Sinusoid, Sine, Gaussian, or sigmoid function, or any combination thereof.

The weighting factors, bias values, and threshold values, or other computational parameters of the neural network, may be “taught” or “learned” in a training phase using one or more sets of training data. For example, the parameters may be trained using the input data from a training data set and a gradient descent or backward propagation method so that the output value(s) that the ANN computes are consistent with the examples included in the training data set. The parameters may be obtained from a back propagation neural network training process.

Any of a variety of neural networks may be suitable for use in analyzing an image of an eye of a subject. Examples can include, but are not limited to, feedforward neural networks, radial basis function networks, recurrent neural networks, residual neural networks, convolutional neural networks, residual convolutional neural networks, and the like, or any combination thereof. In some embodiments, the machine learning makes use of a pre-trained and/or transfer-learned ANN or deep learning architecture. Convolutional and/or residual neural networks can be used for analyzing an image of a subject in accordance with the present disclosure.

For instance, a deep neural network model comprises an input layer, a plurality of individually parameterized (e.g., weighted) convolutional layers, and an output scorer. The parameters (e.g., weights) of each of the convolutional layers as well as the input layer contribute to the plurality of parameters (e.g., weights) associated with the deep neural network model. In some embodiments, at least 100 parameters, at least 1000 parameters, at least 2000 parameters or at least 5000 parameters are associated with the deep neural network model. As such, deep neural network models require a computer to be used because they cannot be mentally solved. In other words, given an input to the model, the model output needs to be determined using a computer rather than mentally in such embodiments. See, for example, Krizhevsky et al., 2012, “Imagenet classification with deep convolutional neural networks,” in Advances in Neural Information Processing Systems 2, Pereira, Burges, Bottou, Weinberger, eds., pp. 1097-1105, Curran Associates, Inc.; Zeiler, 2012 “ADADELTA: an adaptive learning rate method,” CoRR, vol. abs/1212.5701; and Rumelhart et al., 1988, “Neurocomputing: Foundations of research,” ch. Learning Representations by Back-propagating Errors, pp. 696-699, Cambridge, MA, USA: MIT Press, each of which is hereby incorporated by reference.

Neural network models, including convolutional neural network models, suitable for use as models are disclosed in, for example, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference. Additional example neural networks suitable for use as models are disclosed in Duda et al., 2001, Pattern Classification, Second Edition, John Wiley & Sons, Inc., New York; and Hastie et al., 2001, The Elements of Statistical Learning, Springer-Verlag, New York, each of which is hereby incorporated by reference in its entirety. Additional example neural networks suitable for use as models are also described in Draghici, 2003, Data Analysis Tools for DNA Microarrays, Chapman & Hall/CRC; and Mount, 2001, Bioinformatics: sequence and genome analysis, Cold Spring Harbor Laboratory Press, Cold Spring Harbor, New York, each of which is hereby incorporated by reference in its entirety.

Support vector machines. In some embodiments, the model is a support vector machine (SVM). SVM models suitable for use as models are described in, for example, Cristianini and Shawe-Taylor, 2000, “An Introduction to Support Vector Machines,” Cambridge University Press, Cambridge; Boser et al., 1992, “A training algorithm for optimal margin classifiers,” in Proceedings of the 5th Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., pp. 259, 262-265; and Hastie, 2001, The Elements of Statistical Learning, Springer, New York; and Furey et al., 2000, Bioinformatics 16, 906-914, each of which is hereby incorporated by reference in its entirety. When used for classification, SVMs separate a given set of binary labeled data with a hyper-plane that is maximally distant from the labeled data. For cases in which no linear separation is possible, SVMs can work in combination with the technique of ‘kernels’, which automatically realizes a non-linear mapping to a feature space. The hyper-plane found by the SVM in feature space can correspond to a non-linear decision boundary in the input space. In some embodiments, the plurality of parameters (e.g., weights) associated with the SVM define the hyper-plane. In some embodiments, the hyper-plane is defined by at least 10, at least 20, at least 50, or at least 100 parameters and the SVM model requires a computer to calculate because it cannot be mentally solved.

Naïve Bayes models. In some embodiments, the model is a Naive Bayes model. Naïve Bayes models suitable for use as models are disclosed, for example, in Ng et al., 2002, “On discriminative vs. generative classifiers: A comparison of logistic regression and naive Bayes,” Advances in Neural Information Processing Systems, 14, which is hereby incorporated by reference. A Naive Bayes model is any model in a family of “probabilistic models” based on applying Bayes' theorem with strong (naïve) independence assumptions between the features. In some embodiments, they are coupled with Kernel density estimation. See, for example, Hastie et al., 2001, The elements of statistical learning: data mining, inference, and prediction, eds. Tibshirani and Friedman, Springer, New York, which is hereby incorporated by reference.

Nearest neighbor models. In some embodiments, a model is a nearest neighbor model. For nearest neighbors, given a query point x₀ (a test subject), the k training points x₍ᵣ₎, r, . . . , k (here the training subjects) closest in distance to x₀ are identified and then the point x₀ is classified using the k nearest neighbors. Here, the distance to these neighbors is a function of the abundance values of the discriminating gene set. In some embodiments, Euclidean distance in feature space is used to determine distance as d₍ᵢ₎=∥x₍ᵢ₎=x₍₀₎∥. Typically, when the nearest neighbor model is used, the abundance data used to compute the linear discriminant is standardized to have mean zero and variance 1. The nearest neighbor rule can be refined to address issues of unequal class priors, differential misclassification costs, and feature selection. Many of these refinements involve some form of weighted voting for the neighbors. For more information on nearest neighbor analysis, see Duda, Pattern Classification, Second Edition, 2001, John Wiley & Sons, Inc; and Hastie, 2001, The Elements of Statistical Learning, Springer, New York, each of which is hereby incorporated by reference.

A k-nearest neighbor model is a non-parametric machine learning method in which the input consists of the k closest training examples in feature space. The output is a class membership. An object is classified by a plurality vote of its neighbors, with the object being assigned to the class most common among its k nearest neighbors (k is a positive integer, typically small). If k=1, then the object is simply assigned to the class of that single nearest neighbor. See, Duda et al., 2001, Pattern Classification, Second Edition, John Wiley & Sons, which is hereby incorporated by reference. In some embodiments, the number of distance calculations needed to solve the k-nearest neighbor model is such that a computer is used to solve the model for a given input because it cannot be mentally performed.

Random forest, decision tree, and boosted tree models. In some embodiments, the model is a decision tree. Decision trees suitable for use as models are described generally by Duda, 2001, Pattern Classification, John Wiley & Sons, Inc., New York, pp. 395-396, which is hereby incorporated by reference. Tree-based methods partition the feature space into a set of rectangles, and then fit a model (like a constant) in each one. In some embodiments, the decision tree is random forest regression. One specific model that can be used is a classification and regression tree (CART). Other specific decision tree models include, but are not limited to, ID3, C4.5, MART, and Random Forests. CART, ID3, and C4.5 are described in Duda, 2001, Pattern Classification, John Wiley & Sons, Inc., New York, pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests—Random Features,” Technical Report 567, Statistics Department, U.C. Berkeley, September 1999, which is hereby incorporated by reference in its entirety. In some embodiments, the decision tree includes at least 10, at least 20, at least 50, or at least 100 parameters (e.g., weights and/or decisions) and requires a computer to calculate because it cannot be mentally solved.

Regression. In some embodiments, the model uses regression. Regression is any type of regression. For example, in some embodiments, the regression is logistic regression. In some embodiments, the regression is logistic regression with lasso, L2 or elastic net regularization. In some embodiments, those extracted features that have a corresponding regression coefficient that fails to satisfy a threshold value are pruned (removed from) consideration. In some embodiments, a generalization of the logistic regression model that handles multicategory responses is used as the model. Logistic regression is disclosed in Agresti, An Introduction to Categorical Data Analysis, 1996, Chapter 5, pp. 103-144, John Wiley & Son, New York, which is hereby incorporated by reference. In some embodiments, the model makes use of regression disclosed in Hastie et al., 2001, The Elements of Statistical Learning, Springer-Verlag, New York. In some embodiments, the logistic regression includes at least 10, at least 20, at least 50, at least 100, or at least 1000 parameters (e.g., weights) and requires a computer to calculate because it cannot be mentally solved.

Linear discriminant analysis. Linear discriminant analysis (LDA), normal discriminant analysis (NDA), or discriminant function analysis is a generalization of Fisher's linear discriminant, a method used in statistics, pattern recognition, and machine learning to find a linear combination of features that characterizes or separates two or more classes of objects or events. The resulting combination is used as the model (linear model) in some embodiments of the present disclosure.

Mixture model and Hidden Markov model. In some embodiments, the model is a mixture model, such as that described in McLachlan et al., 2002, Bioinformatics 18(3):413-422. In some embodiments, the model is a hidden Markov model such as described by Schliep et al., 2003, Bioinformatics 19(1):i255-i263.

Clustering. In some embodiments, the model is an unsupervised clustering model. In some embodiments, the model is a supervised clustering model. Clustering algorithms suitable for use as models are described, for example, at pages 211-256 of Duda and Hart, Pattern Classification and Scene Analysis, 1973, John Wiley & Sons, Inc., New York, (hereinafter “Duda 1973”) which is hereby incorporated by reference in its entirety. The clustering problem finds groupings in a dataset. To identify these groupings, two issues are addressed. First, a way to measure similarity (or dissimilarity) between two samples is determined. This metric (e.g., similarity measure) is used to ensure that the samples in one cluster are more like one another than they are to samples in other clusters. Second, a mechanism for partitioning the data into clusters using the similarity measure is determined. One way to cluster is to define a distance function and to compute the matrix of distances between all pairs of samples in the training set. If distance is a good measure of similarity, then the distance between reference entities in the same cluster is significantly less than the distance between the reference entities in different clusters. However, there is no absolute requirement that clustering use a distance metric. For example, a nonmetric similarity function s(x, x′) can be used to compare two vectors x and x′. s(x, x′) is a symmetric function whose value is large when x and x′ are somehow “similar.” Once a method for measuring “similarity” or “dissimilarity” between points in a dataset has been selected, clustering uses a criterion function that measures the clustering quality of any partition of the data. Partitions of the data set that extremize the criterion function are used to cluster the data. Particular exemplary clustering techniques used in the present disclosure include, but are not limited to, hierarchical clustering (agglomerative clustering using a nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering. In some embodiments, the clustering comprises unsupervised clustering (e.g., with no preconceived number of clusters and/or no predetermination of cluster assignments).

Ensembles of models and boosting. In some embodiments, an ensemble (two or more) of models is used. In some embodiments, a boosting technique such as AdaBoost is used in conjunction with many other types of learning methods to improve the performance of the model. In this approach, the output of any of the models disclosed herein, or their equivalents, is combined into a weighted sum that represents the final output of the boosted model. In some embodiments, the plurality of outputs from the models is combined using any measure of central tendency known in the art, including but not limited to a mean, median, mode, a weighted mean, weighted median, weighted mode, etc. In some embodiments, the plurality of outputs is combined using a voting method. In some embodiments, a respective model in the ensemble of models is weighted or unweighted.

In some embodiments, the model is a reinforcement learning model. In some embodiments, the reinforcement learning system comprises four main elements—an agent, a policy, a reward signal, and a value function, where the behavior of the agent is defined in terms of the policy. In some embodiments, the reinforcement learning system comprises a learning method. In some implementations, the learning method is an on-policy learning method or an off-policy learning method. On-Policy learning methods evaluate and improve the same policy that is being used to select the agent's actions. Off-policy learning methods evaluate and improve policies that are different from the policy being used for action selection. Reinforcement learning is further described, for example, in Sutton R S, Barto A G, “Reinforcement learning: an introduction,” IEEE Transactions on Neural Networks. 1998; 9(5):1054-1054, which is hereby incorporated herein by reference in its entirety. In some embodiments, the reinforcement learning model includes at least 10, at least 100, at least 1000, at least 10,000, at least 100,000, at least 1×10⁶, at least 1×10⁷, or more parameters. In some embodiments, the reinforcement learning model includes no more than 1×10⁸, no more than 1×10⁷, no more than 1×10⁶, no more than 100,000, no more than 10,000, no more than 1000, or no more than 100 parameters. In some embodiments, the reinforcement learning model consists of from 10 to 1000, from 100 to 100,000, from 10,000 to 1×10⁷, or from 1×10⁶ to 1×10⁸ parameters. In some embodiments, the plurality of parameters for the reinforcement learning model falls within another range starting no lower than 10 parameters and ending no higher than 1×10⁸ parameters.

As used herein, the term “parameter” refers to any coefficient or, similarly, any value of an internal or external element (e.g., a weight and/or a hyperparameter) in a model, regressor, and/or classifier that affects (e.g., modify, tailor, and/or adjust) one or more inputs, outputs, and/or functions in the, model, regressor and/or classifier. For example, in some embodiments, a parameter refers to any coefficient, weight, and/or hyperparameter that is used to control, modify, tailor, and/or adjust the behavior, learning and/or performance of a, model, regressor, and/or classifier. In some instances, a parameter is used to increase or decrease the influence of an input (e.g., a feature) to a model, regressor, and/or classifier. As a nonlimiting example, in some instances, a parameter is used to increase or decrease the influence of a node (e.g., of a neural network), where the node includes one or more activation functions. Assignment of parameters to specific inputs, outputs, and/or functions is not limited to any one paradigm for a given model, regressor, and/or classifier but can be used in any suitable model, regressor, and/or classifier architecture for a desired performance. In some embodiments, a parameter has a fixed value. In some embodiments, a value of a parameter is manually and/or automatically adjustable. In some embodiments, a value of a parameter is modified by a validation and/or training process for a model, regressor, and/or classifier (e.g., by error minimization and/or backpropagation methods, as described elsewhere herein).

In some embodiments, a model, regressor, and/or classifier of the present disclosure comprises a plurality of parameters. In some embodiments the plurality of parameters is n parameters, where: n≥2; n≥5; n≥10; n≥25; n≥40; n≥50; n≥75; n≥100; n≥125; n≥150; n≥200; n≥225; n≥250; n≥350; n≥500; n≥600; n≥750; n≥1,000; n≥2,000; n≥4,000; n≥5,000; n≥7,500; n≥10,000; n≥20,000; n≥40,000; n≥75,000; n≥100,000; n≥200,000; n≥500,000, n≥1×10⁶, n≥5×10⁶, or n≥1×10⁷. In some embodiments n is between 10,000 and 1×10⁷, between 100,000 and 5×10⁶, or between 500,000 and 1×10⁶.

As used herein, the term “instruction” refers to an order given to a computer processor by a computer program. On a digital computer, in some embodiments, each instruction is a sequence of 0s and 1s that describes a physical operation the computer is to perform. Such instructions can include data transfer instructions and data manipulation instructions. In some embodiments, each instruction is a type of instruction in an instruction set that is recognized by a particular processor type used to carry out the instructions. Examples of instruction sets include, but are not limited to, Reduced Instruction Set Computer (RISC), Complex Instruction Set Computer (CISC), Minimal Instruction Set Computers (MISC), Very Long Instruction Word (VLIW), Explicitly Parallel Instruction Computing (EPIC), and One Instruction Set Computer (OISC).

As used herein, “synthon” refers to a representation of a chemical structure having an open valence (attachment bond) at least at one position. In embodiments, synthons are derived from a reagent, from a synthetic reaction sequence, or from the fragmentation of a molecule (e.g., chemical structures derived from the disconnection of a bond). In embodiments, synthons are used to computationally assemble a whole molecule, or when appropriate through synthetic organic chemistry, to synthesize a whole molecule.

### Example Apparatuses for Improving Models for Use in Optimizing Molecular Reactions

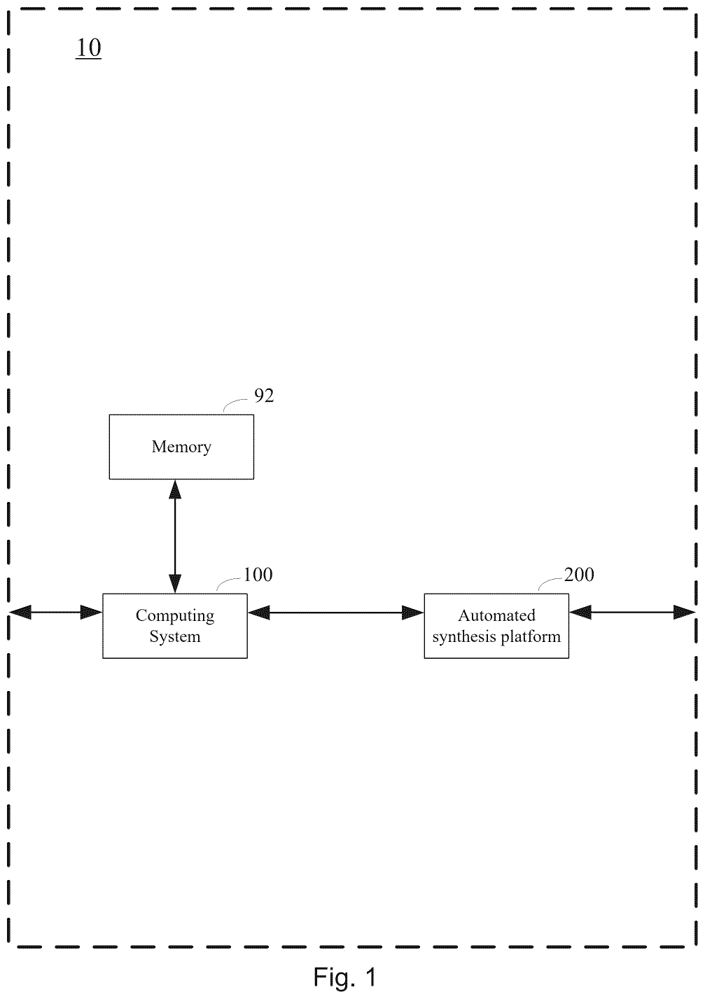

FIG. 1 illustrates an apparatus 10 for improving a first model (e.g., a reinforcement learning model) for use in optimizing a first molecular reaction (e.g., a multistep molecular reaction). In some embodiments, the apparatus of the disclosure comprises computing system 100 and automated synthesis platform 200. Computing system 100 informs automated synthesis platform 200 of inputs (e.g. a first molecular reaction, a second molecular reaction) to perform. Automated synthesis platform 200 informs computing system 100 of inputs (e.g. a first subset of instances, a second subset of instances) to train the model.

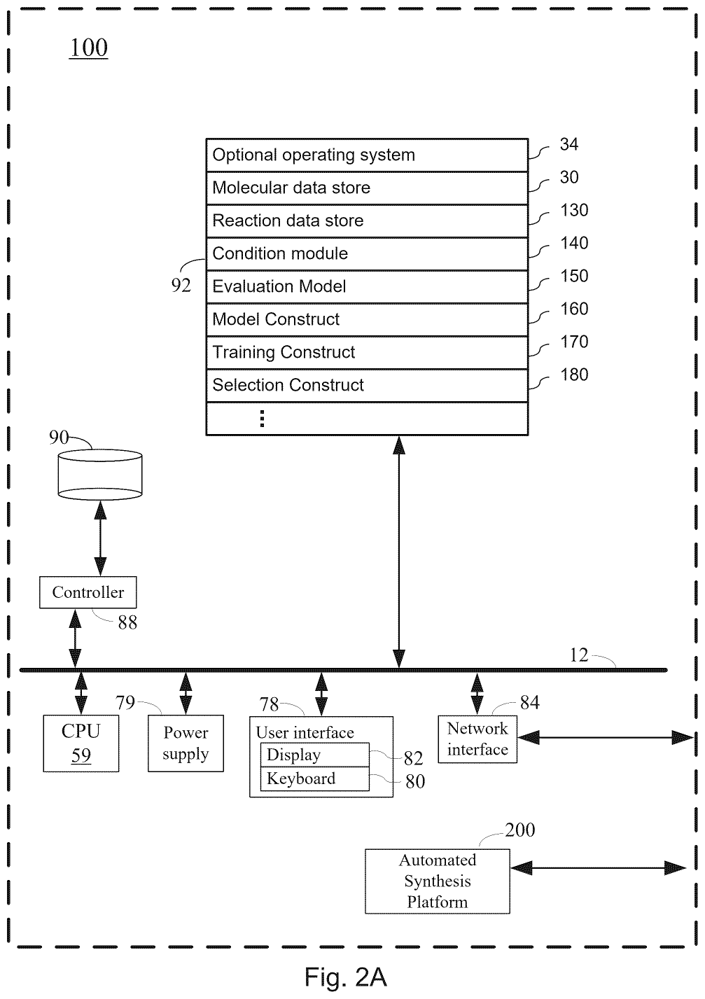

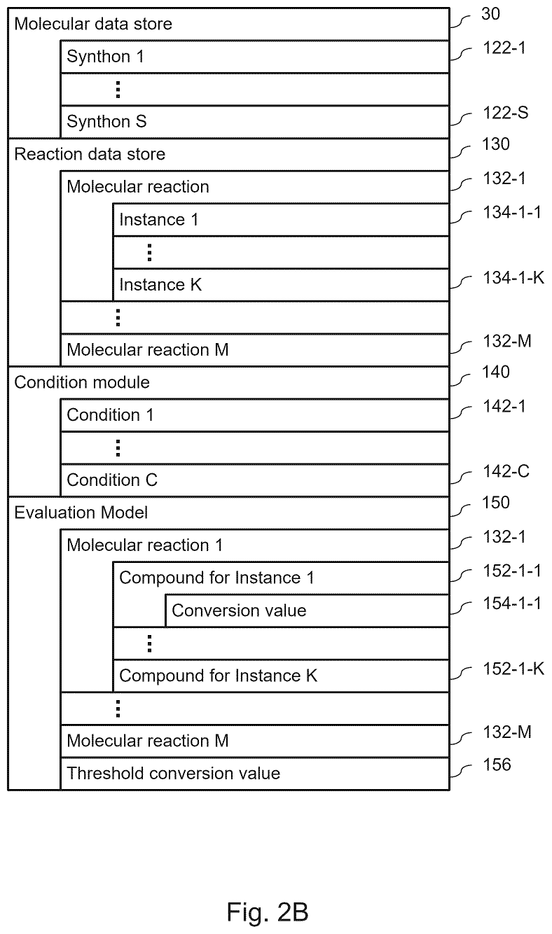

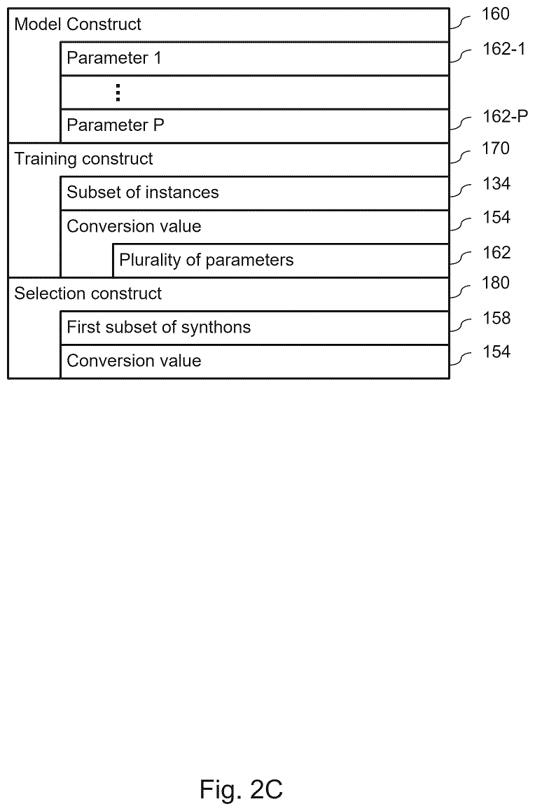

FIGS. 2A-C collectively illustrate a computing system 100 for improving a model (e.g., a reinforcement learning model) for use in optimizing a molecular reaction (e.g., a multistep molecular reaction). In some embodiments, computer system 100 is included in an apparatus of the disclosure.

Referring to FIGS. 2A-C, in some embodiments, computing system 100 comprises one or more computers. For purposes of illustration in FIGS. 2A-C, the computing system 100 is represented as a single computer that includes all of the functionality of the disclosed computing system 100. However, the present disclosure is not so limited. The functionality of the computing system 100 can be spread across any number of networked computers and/or reside on each of several networked computers and/or virtual machines. One of skill in the art will appreciate that a wide array of different computer topologies is possible for the computer system 100 and all such topologies are within the scope of the present disclosure.

The computing system 100 comprises one or more processing units (CPUs) 59, a network or other communications interface 84, an automated synthesis platform 86, a user interface 78 (e.g., including an optional display 82 and optional keyboard 80 or other form of input device), a memory 92 (e.g., random access memory, persistent memory, or combination thereof), one or more magnetic disk storage and/or persistent devices 90 optionally accessed by one or more controllers 88, one or more communication busses 12 for interconnecting the aforementioned components, and a power supply 79 for powering the aforementioned components. To the extent that components of memory 92 are not persistent, data in memory 92 can be seamlessly shared with non-volatile memory 90 or portions of memory 92 that are non-volatile/persistent using known computing techniques such as caching. Memory 92 and/or memory 90 can include mass storage that is remotely located with respect to the central processing unit(s) 59. In other words, some data stored in memory 92 and/or memory 90 may in fact be hosted on computers that are external to computing system 100 but that can be electronically accessed by the computing system 100 over an Internet, intranet, or other form of network or electronic cable using network interface 84. In some embodiments, the computing system 100 makes use of models that are run from the memory associated with one or more graphical processing units in order to improve the speed and performance of the system. In some alternative embodiments, the computing system 100 makes use of models that are run from memory 92 rather than memory associated with a graphical processing unit.

In some embodiments, the memory 92 of the computing system 100 stores:
an optional operating system 34 that includes procedures for handling various basic system services;
a molecular data store 120, optionally comprising a plurality of synthons 122 (e.g., 122-1, . . . 122-S);
a reaction data store 130, optionally comprising one or more molecular reactions 132 (e.g., 132-1, . . . 132-M) and, for a respective selected molecular reaction 132, a plurality of instances 134 (e.g., 134-1-1, . . . 134-1-K);
a condition module 140, optionally comprising a plurality of conditions 142 (e.g., 142-1, . . . 142-C), where:
   the plurality of instances 134 of the molecular reaction 132 (e.g. a first molecular reaction or a second molecular reaction) are performed using the plurality of synthons 122 (e.g., at least 4 synthons) and the plurality of conditions 142 (e.g. an original plurality of normalized conditions), thereby generating a plurality of compounds 152 (e.g., 152-1-1, . . . 152-1-K), and
   for each respective instance 134 of the molecular reaction 132, at least a subset of the plurality of synthons 122 are transformed;
an evaluation module 150, optionally comprising, for each respective instance 134 of the molecular reaction 132, a respective conversion value 154 for the respective instance (e.g., 154-1-1) and a threshold conversion value 156, where:
   a subset of instances 134 are selected from the plurality of instances based on at least the threshold conversion value 156 and the respective conversion value 154 for each respective instance;
a model construct 160, optionally comprising a plurality of parameters 162 (e.g., 162-1, . . . 162-P), where:
   the subset of instances 134 are used to adjust one or more parameters in the plurality of parameters 162 of the model construct 160, thereby obtaining an updated plurality of parameters, and
   the model construct 160 uses, subsequent to obtaining the updated plurality of parameters 162, to search for and identify an updated plurality of normalized conditions 164 for the molecular reaction 132 that collectively have an improved conversion value for the molecular reaction 132 relative to the plurality of conditions 142;
a training construct 170, where:
   the subset of instances 134 are used as independent variables and the respective conversion value 154 for each respective instance are used as dependent variables, and
   the training construct 170 uses the subset of instances 134 as independent variables and the respective conversion value 154 for each respective instance as dependent variables to guide adjustment of one or more parameters in a plurality of parameters 162 of the model construct 160, so that the model construct 160 produces a calculated conversion value in agreement with the corresponding conversion value of each instance of the subset of instances 134 upon input of the subset of instances 134 into the model construct 160; and
a selection construct 180, optionally comprising a first subset of synthons 158, where: the subset of instances 134 are used as independent variables and the respective conversion value 154 for each respective instance are used as dependent variables, and selecting a first subset of synthons that are enriched in the first subset of instances 158 relative to the plurality of instances of the molecular reaction.

In some implementations, one or more of the above identified data elements or modules of the computing system 100 are stored in one or more of the previously mentioned memory devices, and correspond to a set of instructions for performing a function described above. The above identified data, modules, or programs (e.g., sets of instructions) need not be implemented as separate software programs, procedures or modules, and thus various subsets of these modules may be combined or otherwise re-arranged in various implementations. In some implementations, the memory 92 and/or 90 (and optionally 52) optionally stores a subset of the modules and data structures identified above. Furthermore, in some embodiments the memory 92 and/or 90 (and optionally 52) stores additional modules and data structures not described above. In some embodiments, the first neural network 72 is replaced with another form of model.

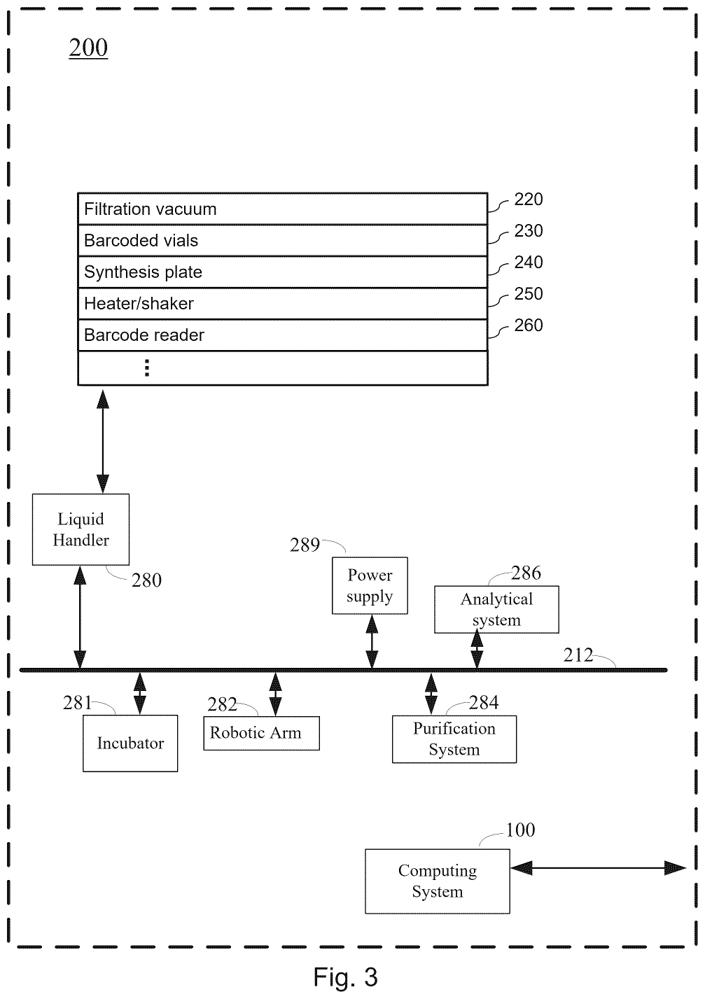

FIG. 3 illustrate an automated synthesis platform 200 for improving a model (e.g., a reinforcement learning model) for use in optimizing a molecular reaction (e.g., a multistep molecular reaction). In some embodiments, automated synthesis platform 200 is included in an apparatus of the disclosure.

Referring to FIG. 3, in some embodiments, automated synthesis platform 200 comprises one or more automated synthesis platforms. For purposes of illustration in FIGS. 2A-C, the automated synthesis platform 200 is represented as a single automated synthesis platform that includes all of the functionality of the disclosed automated synthesis platform 200. However, the present disclosure is not so limited. The functionality of the automated synthesis platform 200 can be spread across any number of networked automated synthesis platform. One of skill in the art will appreciate that a wide array of different automated synthesis platform topologies are possible for the automated synthesis platform 200 and all such topologies are within the scope of the present disclosure.

The automated synthesis platform 200 comprises one or more computing systems 100, a liquid handler 280, an incubator 281, a robotic arm 282, a purification system 284, an analytical system 286; one or more communication busses 212 for interconnecting the aforementioned components, and a power supply 289 for powering the aforementioned components.

In some embodiments, the liquid handler 280 comprises:
a filtration vacuum 220;
one or more barcoded vials 230;
a synthesis plate 240;
a heater/shaker 250; and
a barcode reader 260.

In some embodiments, the apparatus 10 is useful for improving a model for use in an optimization of a molecular reaction. In some embodiments, the apparatus comprises an automated synthesis platform (e.g. automated synthesis platform 200) and a computing system (e.g. computing system 100). In some embodiments, the computing system comprises one or more processors and memory addressable by the one or more processors, the memory storing the model. In some embodiments, the computing system 100 informs the automated synthesis platform 200 of the molecular reaction (see, for example, FIG. 1).

In some embodiments, the molecular reaction is a multistep molecular reaction.

In some embodiments, the molecular reaction comprises at least 2, at least 3, or at least 4 steps. In some embodiments, the molecular reaction comprises at least 5, at least 10, at least 20, or at least 30 steps. In some embodiments, the molecular reaction comprises no more than 50, no more than 30, no more than 20, no more than 10, or no more than 5 steps. In some embodiments, the molecular reaction consists of from 2 to 5, from 2 to 10, from 5 to 20, from 10 to 30, or from 20 to 50 steps. In some embodiments, the molecular reaction falls within another range starting no lower than 2 steps and ending no higher than 50 steps.

In some embodiments, the molecular reaction is a first molecular reaction (e.g., a molecular reaction type) selected from a plurality of molecular reactions (e.g., a plurality of molecular reaction types). In some embodiments, the molecular reaction is a second molecular reaction (e.g., a molecular reaction type) selected from a plurality of molecular reactions (e.g., a plurality of molecular reaction types).

In some embodiments, the plurality of molecular reactions comprises at least 2, at least 5, at least 10, at least 50, at least 100, at least 500, or at least 1000 molecular reactions. In some embodiments, the plurality of molecular reactions comprises no more than 5000, no more than 1000, no more than 100, no more than 50, or no more than 20 molecular reactions. In some embodiments, the plurality of molecular reactions consists of from 10 to 100, from 50 to 200, from 100 to 500, or from 500 to 5000 molecular reactions. In some embodiments, the plurality of molecular reactions falls within another range starting no lower than 2 molecular reactions and ending no higher than 5000 molecular reactions.

In some embodiments, the plurality of molecular reactions comprises one or more reaction SMILES (Simplified Molecular Input Line Entry Specification). SMILES representations comprise at least two fundamental types of symbols for atoms and bonds, respectively. These symbols are used to specify a molecular graph for a respective molecule (e.g., using “nodes” and “edges”) and assign labels to the components of the graph that indicate, for example, the type of atom each node represents and/or the type of bond each edge represents.

In some embodiments, the plurality of molecular reactions comprises one or more reaction SMARTS (SMILES arbitrary target specification). SMARTS refers to a language that allows for the specification of molecular substructures using an extended set of rules. In particular, SMARTS uses atomic and bond symbols to specify a molecular graph, where the labels for the graph's nodes and edges (e.g., “atoms” and “bonds”) are extended to include “logical operators” and special atomic and bond symbols, thus allowing SMARTS atoms and bonds to be more general. Moreover, the SMARTS language can be used for the expression of molecular reactions (e.g., “reaction queries”). In some implementations, reaction queries are composed of optional reactant, agent, and product parts, which are separated by a “>” character. In such cases, the components of a reaction query match the corresponding roles within the reaction target. SMILES and SMARTS reactions are further disclosed, for example, in “SMARTS Theory Manual,” Daylight Chemical Information Systems, Santa Fe, New Mexico, available on the Internet at daylight.com/dayhtml/doc/theory/theory.smarts.html, which is hereby incorporated herein by reference in its entirety.

In some embodiments, the plurality of molecular reactions includes, but is not limited to, named reactions, organic synthesis reactions, protecting groups (see, Green and Wuts, Protective Groups in Organic Synthesis, second edition, John Wiley & Sons, Inc., New York, 1991, which is hereby incorporated by reference), total synthesis, Flow Chemistry, Green Chemistry, Microwave Synthesis, Multicomponent Reactions, Organocatalysis, and/or Sonochemistry. Alternatively or additionally, in some embodiments, the plurality of molecular reactions includes, but is not limited to, esterification reactions (e.g., methyl esterification), hydrolysis of esters, amide synthesis, transamidation, oxidative amidation, nucleophilic aromatic substitution reactions, protecting group addition/removal reactions (e.g., additional/removal of tert-butoxycarbonyl protecting group (BOC group)); addition/removal of silyl protective group (e.g., trimethylsilyl group, triethylsilyl group, tert-butyldimethylsilyl (TBDMS), tert-butyldiphenylsilyl group (TBDPS)), reaction of electrophiles with amines, synthesis of heterocycles, reductive amination, debenzylation, alkylation of an alcohol (e.g., phenol), sulfonamide formation, reduction (e.g., reduction of nitro group to amine group, reduction of aldehyde, ketone, carboxylic acid, etc., to alcohol), oxidation (e.g., oxidation of an alcohol to an aldehyde, ketone, carboxylic acid, etc.), diazotization followed by reaction with nucleophile, lithiation reaction (e.g., aryl lithiation) followed by reaction with electrophile, halogenation (e.g., aromatic halogenation, aldol reaction, oxidation/reduction of olefin, hydrogenation, oxygenation/deoxygenation, oxidative cleavage reactions, alkylation, hydrolysis and/or decarboxylation of beat-keto ester, Schmidt Reaction, Schotten-Baumann Reaction, Ugi Reaction, arylamine synthesis, Grignard reaction, Buchwald-Hartwig Reaction, Chan-Lam Coupling, Petasis Reaction, Ullmann Reaction, Hiyama Coupling, Kumada Coupling, Miyaura Borylation Reaction, Negishi Coupling, Stille Coupling, Suzuki-Miyaura Coupling, Sonogashira Coupling, Click Chemistry, cycloaddition reactions including but not limited to Azide-Alkyne Cycloaddition, Copper-Catalyzed Azide-Alkyne Cycloaddition (CuAAC), Ruthenium-Catalyzed Azide-Alkyne Cycloaddition (RuAAC), Huisgen 1,3-Dipolar Cycloaddition, and Synthesis of 1,2,3-Triazoles, Wittig reaction, Horner-Wadsworth-Emmons reaction, epoxide synthesis, Jacobsen-Katsuki Epoxidation, Prilezhaev Reaction, Sharpless Epoxidation, Shi Epoxidation, and/or ring opening reactions of epoxides. Various molecular reactions are known in the art and are contemplated for use in the present disclosure. For instance, non-limiting examples of molecular reactions are further described in the Organic Chemistry Portal, available on the Internet at organic-chemistry.org.

### Compound Synthesis.

In some embodiments, the optimization further includes performing a plurality of instances 134 of the first molecular reaction 132 using a first plurality of synthons 122 (e.g., at least 4 synthons) and an original plurality of normalized conditions 142, comprising, for each respective instance 134 of the first molecular reaction 132, transforming, with the automated synthesis platform (e.g. automated synthesis platform 200) at least a subset of the plurality of synthons 122 using the first molecular reaction, thereby generating a first plurality of compounds 152. For example, in some such embodiments, the first plurality of compounds is generated over the plurality of instances of the first molecular reaction. In some embodiments, each respective instance of the first molecular reaction generates a compound. In some embodiments, each respective instance of the first molecular reaction generates a first plurality of compounds.

In some embodiments, the plurality of synthons comprises at least 2, at least 4, at least 5, at least 10, at least 20, at least 50, at least 100, at least 500, at least 1000, at least 10,000, or at least 100,000 synthons. In some embodiments, the plurality of synthons comprises no more than 1×10⁶, no more than 100,000, no more than 10,000, no more than 1000, no more than 100, or no more than 50 synthons. In some embodiments, the plurality of synthons consists of from 2 to 20, from 10 to 100, from 50 to 1000, from 500 to 10,000, from 2000 to 500,000, or from 100,000 to 1×10⁶ synthons. In some embodiments, the plurality of synthons falls within another range starting no lower than 2 synthons and ending no higher than 1×10⁶ synthons.

In some embodiments, the plurality of compounds comprises at least 2 or more, at least 4 or more, at least 5 or more, at least 10 or more, at least 20 or more, at least 50 or more, at least 100 or more, at least 500 or more, at least 1000 or more, at least 10,000, or at least 100,000 compounds. In some embodiments, the plurality of compounds comprises 100 or more, 500 or more, 1000 or more, 2000 or more or 10,000 or more compounds. In some embodiments, the plurality of compounds comprises no more than 1×10⁶, no more than 100,000, no more than 10,000, no more than 1000, no more than 100, or no more than 50 compounds. In some embodiments, the plurality of compounds consists of from 2 to 20, from 10 to 100, from 50 to 1000, from 500 to 10,000, from 2000 to 500,000, or from 100,000 to 1×10⁶ compounds.

In some embodiments, each compound in the plurality of compounds satisfies two or more rules, three or more rules, or all four rules of the Lipinski's rule of Five: (i) not more than five hydrogen bond donors, (ii) not more than ten hydrogen bond acceptors, (iii) a molecular weight under 500 Daltons, and (iv) a Log P under 5.

In some embodiments, each compound in the plurality of compounds is an organic compound having a molecular weight of less than 500 Daltons, less than 1000 Daltons, less than 2000 Daltons, less than 4000 Daltons, less than 6000 Daltons, less than 8000 Daltons, less than 10000 Daltons, or less than 20000 Daltons. In some embodiments, each compound in the first plurality of compounds is an organic compound having a molecular weight of between 400 Daltons and 10000 Daltons.

In some embodiments, the plurality of normalized conditions comprises at least 10, at least 50, at least 100, at least 500, at least 1000, at least 10,000, at least 100,000, at least 1×10⁶, at least 1×10⁷, or at least 1×10⁸ normalized conditions. In some embodiments, the plurality of normalized conditions comprises no more than 1×10⁹, no more than 1×10⁸, no more than 1×10⁷, no more than 1×10⁶, no more than 100,000, no more than 10,000, no more than 1000, no more than 100, or no more than 50 normalized conditions. In some embodiments, the plurality of normalized conditions consists of from 10 to 1000, from 500 to 100,000, from 100,000 to 1×10⁶, from 1×10⁶ to 1×10⁸, or from 1×10⁷ to 1×10⁹ normalized conditions. In some embodiments, the plurality of normalized conditions falls within another range starting no lower than 10 normalized conditions and ending no higher than 1×10⁹ normalized conditions.

In some embodiments, each respective normalized condition in the plurality of normalized conditions is selected from the group consisting of: synthon type, reactant type, reagents, catalysts, solvents, concentrations, order of addition, amount of equivalents for addition, synthon scope, temperature, incubation and/or reaction time, stoichiometry of synthons, stoichiometry of reactants, and/or stoichiometry of reagents. In some embodiments, one or more reagents are synthetic equivalents of a synthon. As used herein, “synthetic equivalent” refers to a reactant that carries out the function of a synthon.

Alternatively or additionally, in some embodiments, a normalized condition in the plurality of normalized conditions is an experimental layout (e.g., on a reaction plate). In some embodiments, the molecular reaction, and/or one or more instances thereof, is performed in a reaction plate, including, but not limited to, a 12-well, 24-well, 48-well, 96-well, and/or 384-well plate.

In some embodiments, a normalized condition in the plurality of normalized conditions is one or more solvents suitable for use in automation. In some embodiments, a solvent having a boiling point, rate of evaporation, density, and/or surface tension the same or substantially the same or greater than that of water would be suitable for use in automation, whereas a solvent having a boiling point, a rate of evaporation, density, and/or surface tension less than that of water would not be ideal for use in automation. In some embodiments, the molecular reaction, and/or one or more instances thereof, is performed using one or more solvents suitable for use in automation, including but not limited to N-methyl-2-pyrrolidone (NMP), dimethylformamide (DMF), acetonitrile (MeCN), dimethyl sulfoxide (DMSO), or mixtures thereof). A non-limiting example of a solvent not ideal for use in automation is methylene chloride (DCM). In some embodiments, a solvent suitable for automation is a solvent capable of solubilizing one or more components of a reaction (e.g., reactants, reagents, catalysts) and/or exhibits thermal stability when heated during a reaction, including but not limited to N-methyl-2-pyrrolidone (NMP).

In some embodiments, each respective instance of the molecular reaction refers to an implementation, replicate, and/or “run” of the molecular reaction. In some embodiments, a first instance of the molecular reaction is performed as a replicate of a second instance of the molecular reaction, where both the first and the second instance of the molecular reaction have the same synthons and/or the same normalized conditions. In some embodiments, a first instance of the molecular reaction and a second instance of the molecular reaction are performed having a different set of synthons and/or a different set of normalized conditions. In some embodiments, each respective instance of the molecular reaction has a different set of synthons and/or a different set of normalized conditions from any other instance of the molecular reaction.

In some embodiments, each instance (e.g., each run) of the molecular reaction is performed using a different set of conditions (for instance, to test which conditions result in improved conversion values by permutating the different reaction conditions under which the molecular reaction is performed). In some embodiments, the different sets of conditions include one or more different synthons (e.g., selected to be used as starting components for the molecular reaction), and/or one or more different normalized conditions (e.g., reaction conditions such as temperature, incubation time, concentrations, etc., as described above) used to produce a compound.

Moreover, in some implementations, for each instance of the molecular reaction, a set of normalized conditions under which the molecular reaction is to be performed is generated by a model. In some embodiments, the normalized conditions are generated by the model responsive to inputting the plurality of synthons (e.g., a plurality of building blocks or starting components upon which the molecular reaction is performed). In some embodiments, the method further includes inputting, into the model, an indication of the selected molecular reaction.

In some embodiments, the model is trained to optimize the molecular reaction by generating improved normalized conditions used in performing the molecular reaction. As described in further detail below, such output is improved through a training process in which the parameters of the model are adjusted based on an evaluation of the compounds produced according to the outputted normalized conditions, where the evaluation includes a comparison of an evaluation metric (e.g., a conversion value) for the compound against a threshold evaluation metrics (e.g., a threshold conversion value). Further improvement of the model occurs, in some embodiments, through subsequent iterations of compound generation, evaluation, and adjustment of model parameters.

In some embodiments, the optimization further includes, for each respective synthon in the subset of the plurality of synthons: selecting one or more reactants, in a plurality of reactants, that are synthetic equivalents of the respective synthon, thereby obtaining a subset of the plurality of reactants (e.g. a “synthetic equivalent” refers to a reactant that carries out the function of a synthon). In some such embodiments, the performing the plurality of instances of the molecular reaction transforms, for each respective instance of the molecular reaction, at least the subset of the plurality of reactants using the molecular reaction.

In some embodiments, each respective instance in the plurality of instances of the molecular reaction comprises a respective subset of reactants in the plurality of reactants.

In some embodiments, the optimization further includes, prior to the performing a first plurality of instances of the first molecular reaction, selecting the plurality of synthons from a plurality of initial synthons based upon the molecular reaction. In other words, in some embodiments, synthons are selected based on the type of molecular reaction selected. For example, in some implementations, the plurality of synthons is identified as those upon which the molecular reaction can be performed, based on one or more factors (e.g., primary, secondary, benzyl, and/or aryl substitutions, sterically hindered versus available, electron withdrawing versus electron donating groups, etc.).

In some embodiments, a reaction database, such as Reaxys, is used to identify the synthons and/or normalized conditions used for each instance of the molecular reaction. In some implementations, the synthons and/or normalized conditions are selected by selecting the most common reagents used for the respective molecular reaction and/or reagents that are commercially available. In some implementations, this is an automated consolidation of reagents, catalysts, solvents, etc., from such a database. Alternatively or additionally, in some embodiments, the selection of the plurality of synthons is performed manually, for instance by reviewing literature and choosing synthons and/or normalized conditions that appear repeatedly in the literature. However, the manual process can be time consuming and limited in the number of examples that can be considered.

In some embodiments, the plurality of initial synthons comprises at least 1000, at least 5000, at least 10,000, at least 100,000, at least 1×10⁶, at least 1×10⁷, at least 1×10⁸, at least 1×10⁹, at least 1×10¹⁰, at least 1×10¹¹, or at least 5×10¹¹ initial synthons. In some embodiments, the plurality of initial synthons comprises no more than 1×10¹², no more than 1×10¹¹, no more than 1×10¹⁰, no more than 1×10⁹, no more than 1×10⁸, no more than 1×10⁷, no more than 1×10⁶, no more than 100,000, or no more than 10,000 initial synthons. In some embodiments, the plurality of initial synthons consists of from 1000 to 100,000, from 10,000 to 1×10⁷, from 1×10⁶ to 1×10⁸, from 1×10⁸ to 1×10¹¹, or from 1×10⁹ to 1×10¹² initial synthons. In some embodiments, the plurality of initial synthons falls within another range starting no lower than 1000 initial synthons and ending no higher than 1×10¹² initial synthons.

In some embodiments, the plurality of synthons comprises a first subplurality of synthons and a second subplurality of synthons, where each respective synthon in the first subplurality of synthons is capable of reacting with each respective synthon in the second subplurality of synthons to generate a plurality of compounds. In some embodiments, at least one of each respective synthon in the first subplurality of synthons and at least one synthon of each respective synthon in the second subplurality of synthons are ionic (charged) synthons. In some embodiments, at least one of each respective synthon in the first subplurality of synthons is a donor synthon (e.g., a nucleophilic or negatively charged synthon) and at least one synthon of each respective synthon in the second subplurality of synthons is an acceptor synthon (e.g., an electrophilic or positively-charged synthon), wherein the donor synthon is capable of reacting with the acceptor synthon to generate a compound. In a non-limiting example, for an amide reaction, at least one of each respective synthon in the first subplurality of synthons is a donor synthon comprising at least one negatively-charged amine, and at least one synthon of each respective synthon in the second subplurality of synthons comprising at least one positively charged carbon of a carbonyl group, wherein the donor synthon is capable of reacting with the acceptor synthon to generate an amide compound (see, for example, Scheme A).

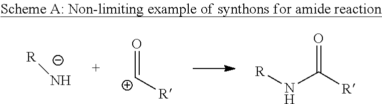

In some embodiments, at least one of each respective synthon in the first subplurality of synthons and at least one synthon of each respective synthon in the second subplurality of synthons are each neutral (uncharged) synthons. In a non-limiting example, for cycloaddition reaction, at least one of each respective synthon in the first subplurality of synthons is a neutral synthon comprising at least one diene, and at least one of each respective synthon in the second subplurality of synthons is a neutral synthon comprising at least one alkene, wherein the neutral synthons are capable of reacting to generate a ring (see, for example, Scheme B).

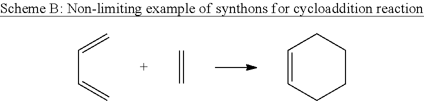

In some embodiments, the plurality of synthons comprises a first subplurality of synthons and a second subplurality of synthons, where each respective synthon in the first subplurality of synthons is a first reactant and each respective synthon in the second subplurality of synthons is a second reactant. For each respective step in the multistep molecular reaction, the respective step transforms a first reactant selected from the first subplurality and a second reactant selected from the second subplurality of synthons. In some embodiments, the chemical structure of a first reactant comprises and/or is the same or substantially the same as the chemical structure of one or more synthons in the first plurality of synthons. In some embodiments, the chemical structure of a second reactant comprises and/or is the same or substantially the same as the chemical structure of one or more synthons in the second plurality of synthons. In some embodiments, a first reactant is a synthetic equivalent of one or more synthons in the first plurality of synthons. In some embodiments, a second reactant is a synthetic equivalent of one or more synthons in the second plurality of synthons.

In some embodiments, a reactant (e.g., a first reactant and/or a second reactant) is selected based on one or more factors selected from (a)-(g):
(a) Type of valence bond (e.g., single bond, double bond, triple bond);
(b) Electronics of reactants (e.g., reactant is or comprises electron withdrawing moiety such as lower alkenyl, lower alkynyl, aryl, aldehyde (—COH), acyl (—COR), carbonyl (—CO), carboxylic acid (—COOH), ester (—COOR), halide (—Cl, —F, —Br, —I), haloalkyl, cyano (—CN), sulfoxide (—SOR), sulfonyl (—SO₂R), sulfonic acid (—SO₃H), and primary, secondary, or tertiary ammonium (—NR₃+), and nitro (—NO₂), or reactant is or comprises electron donating moiety such as hydroxyl (—OR), lower alkoxy (including methoxy, ethoxy, and the like), lower alkyl (including methyl, ethyl, and the like), amino, lower alkylamino, di(lower alkyl)amino, aryloxy (e.g., phenoxy), mercapto, lower alkylthio, lower alkylmercapto, disulfide (e.g., lower alkyldithio).
(c) Structure of reactive moiety (e.g., primary, secondary, or tertiary structure);
(d) Sterics (e.g., the reactant comprises (i) a sterically hindered moiety (e.g., t-butyl group), optionally wherein the sterically hindered moiety is present at the reactive portion of the reactant and/or 1, 2, 3, or more than 3 atoms are present between the sterically hindered moiety and the reactive portion of the reactant, or (ii) the reactant does not comprise a sterically hindered moiety at the reactive portion of the reactant and/or within 1, 2, 3, or more than 3 atoms from the reactive portion of the reactant);
(e) Substituents of the reactant (e.g., a reactant is selected because it comprises a substituent useful in the molecular reaction and/or useful in a subsequent molecular reaction of a multistep molecular reaction);
(f) Commercial availability of the reactant; and
(g) Cost of the reactant (e.g., cost to purchase and/or synthesize the reactant).

In some embodiments, for each respective instance in the plurality of instances of the molecular reaction, for at least a first step in the molecular reaction, the plurality of synthons consists of a first subplurality of n synthons and a second subplurality of k synthons arranged in an n by k grid, and the subset of the plurality of synthons transformed by the molecular reaction comprises (i) one or more synthons selected from the first subplurality of synthons and (ii) one or more synthons selected from the second subplurality of synthons.

In some embodiments, n is at least 2, at least 4, at least 5, at least 10, at least 20, at least 50, at least 100, or at least 500. In some embodiments, n is no more than 1000, no more than 500, no more than 100, no more than 50, no more than 20, or no more than 10. In some embodiments, n is from 2 to 10, from 4 to 30, from 20 to 100, from 80 to 500, or from 300 to 1000. In some embodiments, n falls within another range starting no lower than 2 and ending no higher than 1000.

In some embodiments, k is at least 2, at least 4, at least 5, at least 10, at least 20, at least 50, at least 100, or at least 500. In some embodiments, k is no more than 1000, no more than 500, no more than 100, no more than 50, no more than 20, or no more than 10. In some embodiments, k is from 2 to 10, from 4 to 30, from 20 to 100, from 80 to 500, or from 300 to 1000. In some embodiments, k falls within another range starting no lower than 2 and ending no higher than 1000.

In some embodiments, n and k are positive integer values. In some embodiments, n and k have the same or different values.

In some embodiments, n is between 2 and 8 and k is between 4 and 12. In some embodiments, n is between 6 and 20 and k is between 15 and 40.

In some embodiments, for each respective instance in the plurality of instances of the molecular reaction, the plurality of synthons comprises at least a first subplurality of synthons and a second subplurality of synthons, a first step in the molecular reaction samples one or more synthons from the first subplurality of synthons, and a second step in the molecular reaction samples one or more synthons from the second subplurality of synthons. In some implementations, each step of the multistep molecular reaction is performed by sampling from a different subset of synthons in the plurality of synthons. In some embodiments, each of at least a first step of the multistep molecular reaction is performed by sampling from the same subset of synthons in the plurality of synthons as a second step of the multistep molecular reaction.

In some embodiments, one or more filtering steps are performed after one or more steps of the multistep molecular reaction are performed. In a non-limiting example, a filtering step includes filtration of the molecular reaction sample, which is useful to remove solid impurities and/or to isolate an organic solid. Any filtration method is contemplated by the present disclosure, including but not limited to gravity filtration, vacuum filtration, and suction filtration. In some embodiments, one or more filtering steps are performed after each of at least a first step of a multistep molecular reaction and before each of at least a second step of a multistep molecular reaction. In some embodiments, the filtration step is performed using filtration vacuum 220.

In some embodiments, a respective subplurality of synthons in the plurality of synthons comprises at least 2, at least 4, at least 5, at least 10, at least 20, at least 50, at least 100, or at least 500 synthons. In some embodiments, a respective subplurality of synthons in the plurality of synthons comprises no more than 1000, no more than 500, no more than 100, no more than 50, no more than 20, or no more than 10 synthons. In some embodiments, a respective subplurality of synthons in the plurality of synthons consists of from 2 to 10, from 4 to 30, from 20 to 100, from 80 to 500, or from 300 to 1000 synthons. In some embodiments, a respective subplurality of synthons in the plurality of synthons falls within another range starting no lower than 2 synthons and ending no higher than 1000 synthons.

In some embodiments, the plurality of instances of the molecular reaction comprises at least 1×10⁶ instances.

In some embodiments, the plurality of instances of the molecular reaction comprises at least 1000, at least 10,000, at least 100,000, at least 1×10⁶, at least 1×10⁷, at least 1×10⁸, or at least 1×10⁹ instances. In some embodiments, the plurality of instances of the molecular reaction comprises no more than 1×10¹⁰, no more than 1×10⁹, no more than 1×10⁸, no more than 1×10⁷, no more than 1×10⁶, no more than 100,000, or no more than 10,000 instances. In some embodiments, the plurality of instances of the molecular reaction consists of from 1000 to 100,000, from 10,000 to 1×10⁶, from 1×10⁶ to 1×10⁸, or from 1×10⁸ to 1×10¹⁰ instances. In some embodiments, the plurality of instances of the molecular reaction falls within another range starting no lower than 1000 instances and ending no higher than 1×10¹⁰ instances.

In some embodiments, the performing a plurality of instances of the molecular reaction comprises, for each respective instance of the molecular reaction, transforming the subset of the plurality of synthons using the molecular reaction using an automated synthesis platform (e.g., automated synthesis platform 200), thereby generating the plurality of compounds. In some embodiments, the automated synthesis platform comprises an automated synthesis robot.

Generally, performing chemistry on automation can differ from manually performed chemistry. Automated chemistry reduces the need for individual labor and training, with the added advantage of standardizing experiments and data read outs. Variables such as human error, time of day, order of addition of chemicals, laboratory temperature can lead to varying data outputs even when using common workflows. Conversely, due to the high number of reactions performed during automated chemistry, automated approaches are sensitive to the conditions or synthons in the reaction in order to achieve successful synthesis. Having a low conversion rate in any of the steps in a multistep reaction can impact later steps if there is insufficient yield to continue the reaction process, resulting in greater expenses, wasted resources, and slower device or apparatus runs. Compounding such issues is the fact that all molecules are different, with different synthetic routes, starting materials, electronics, sterics, and so on. Accordingly, one goal in automating many reactions is the ability to identify the best chemistry conditions to apply to specific building blocks within a given reaction type, and/or to determine whether a particular molecular reaction is automatable or not across a range of possible synthons and conditions.

In some embodiments, the automated synthesis platform comprises one or more instruments selected from the group consisting of: liquid handlers, shakers, heaters, robotic arms, decappers, plate sealers, barcode readers, and/or analyzers.

In some embodiments, the automated synthesis platform comprises an integration module to integrate the one or more instruments. In some embodiments, the integration module comprises one or more integration software tools for scheduling, control, and/or automation of the one or more instruments.

Various automated synthesis platforms and integration modules are contemplated for use in the present disclosure, as will be apparent to one skilled in the art.

### Evaluation of Conversion.

In some embodiments, the optimization further includes obtaining, for each respective instance 134 of the first molecular reaction 132, a respective conversion value 154 for the respective instance.

In some embodiments, the respective conversion value for the respective instance is determined as a percent yield of a corresponding compound obtained for the respective instance of the molecular reaction. In some embodiments, the respective conversion value is a percent yield of a corresponding compound obtained for the respective instance of the molecular reaction determined as a ratio of product to starting material. In some embodiments, the respective conversion value is determined based on the mol percent (mol %) of a corresponding compound obtained for the respective instance of the molecular reaction determined as a ratio of moles of product to moles of starting material. In some embodiments, the respective conversion value is determined as a percent of a remaining amount (e.g., mass, volume) of one or more synthons and/or one or more reactants (e.g., a first reactant and/or a second reactant) obtained for the respective instance of the molecular reaction. For instance, in an example embodiment, where 80% of a first synthon and/or a first reactant is consumed in the respective instance of the molecular reaction, the percent of a remaining amount of the first synthon and/or the first reactant is 20%. In some embodiments, the respective conversion value is determined based on the mol percent (mol %) of one or more synthons and/or one or more reactants (e.g., a first reactant and/or a second reactant) obtained for the respective instance of the molecular reaction. In some embodiments, the respective conversion value is determined as a percent consumption (e.g., mass, volume) of one or more synthons and/or one or more reactants (e.g., a first reactant and/or a second reactant) obtained for the respective instance of the molecular reaction. For instance, in an example embodiment, where 80% of a first synthon and/or a first reactant is consumed in the respective instance of the molecular reaction, the percent consumption of the first synthon and/or the first reactant is 80%. In some embodiments, the respective conversion value is calculated using actual values of the corresponding compound and/or one or more synthons and/or one or more reactants (e.g. by directly measuring the remaining amounts after synthesis of the corresponding compound). In some embodiments, the respective conversion value is calculated using estimated values of the corresponding compound and/or one or more synthons and/or one or more reactants (e.g. by using methods capable of determining concentrations such as UV spectroscopy).

In some embodiments, the optimization further includes selecting a first subset of instances from the first plurality of instances 134 based on at least a first threshold conversion value 156 for the respective conversion value 154 of each respective instance. In some embodiments, the automated synthesis platform (e.g., automated synthesis platform 200) informs the computing system of the first subset of instances.

In some embodiments, the respective conversion value is a percent yield of a corresponding compound obtained for the respective instance of the molecular reaction determined as a ratio of product to starting material, where the threshold conversion value is at least about 20%.

In some embodiments, the respective conversion value is a percent yield of a corresponding compound obtained for the respective instance of the molecular reaction determined as a percent of amount (e.g., mass, volume) of expected product to the actual amount of product produced in the respective instance of the molecular reaction. In some embodiments, the threshold conversion value is at least about 20%, at least about 30%, at least about 40%, at least 5 about 0%, at least about 60%, at least about 70%, at least about 80%, at least about 85%, at least about 90%, or at least about 95%.

In some embodiments, the threshold conversion value is at least about 40%, at least about 50%, or at least about 60%. In some embodiments, the threshold conversion value is at least about 10%, at least about 20%, at least about 30%, at least about 40%, at least about 50%, at least about 60%, at least about 65%, at least about 70%, at least about 75%, at least about 80%, at least about 85%, or at least about 90%. In some embodiments, the threshold conversion value is no more than about 95%, no more than about 90%, no more than about 80%, no more than about 70%, no more than about 60%, no more than about 50%, no more than about 40%, no more than about 30%, or no more than about 20%. In some embodiments, the threshold conversion value is from about 10% to about 30%, from about 20% to about 50%, from about 30% to about 60%, from about 40% to about 80%, from about 50% to about 90%, or from about 70% to about 95%. In some embodiments, the threshold conversion value falls within another range starting no lower than about 10% and ending no higher than about 95%.

In some embodiments, the conversion value of a compound generated in each respective instance of the molecular reaction is compared to the threshold conversion value. In some embodiments, when the conversion value meets or exceeds the threshold conversion value, the respective instance of the molecular reaction is selected for retention in the subset of instances, and when conversion value does not meet the threshold conversion value, the respective instance of the molecular reaction is not selected for the subset of instances.

In some embodiments, a respective instance is labeled with an indication of conversion based on the comparison of the conversion value of the respective compound against the threshold conversion value. In some embodiments, the indication of conversion is selected from the group consisting of fail, success, and/or intermediate. In some embodiments, the indication of conversion comprises a shading or a color (e.g., red for fail, green for success, yellow for intermediate success, and/or orange for intermediate fail). Other methods of indicating conversion are possible, as will be apparent to one skilled in the art.

### Training of Model.

In some embodiments, the optimization further includes training the first model by using i) the first subset of instances 134 as independent variables and ii) the corresponding conversion value of each instance of the first subset of instances 134 as dependent variables, to guide adjustment of one or more parameters in a plurality of parameters of the first model, so that the first model produces a calculated conversion value in agreement with the corresponding conversion value of each instance of the subset of instances upon input of the subset of instances 134 into the first model. For example, in some embodiments, the independent variable is a subset of reaction conditions and/or synthons, wherein each instance is or comprises one or more reaction conditions and/or synthons used in a reaction well.

In some embodiments, the model is a graph neural network. In some embodiments, the graph neural network is pre-trained, prior to the training v), on a local level using a plurality of unlabeled molecules. The plurality of unlabeled molecules is other than the plurality of compounds. In some embodiments, the plurality of unlabeled molecules is the ZINC15 database. Additional examples of graph neural networks are disclosed in, for example, Hu et al., “Strategies for Pre-training Graph Neural Networks,” arXiv 2019, which is hereby incorporated by reference in its entirety.

In some embodiments, the plurality of unlabeled molecules comprises 1000 or more unlabeled molecules or 10,000 or more unlabeled molecules. In some embodiments, the plurality of unlabeled molecules comprises 100 or more unlabeled molecules, 500 or more unlabeled molecules, 1000 or more unlabeled molecules, 5000 or more unlabeled molecules, 10,000 or more unlabeled molecules, 25,000 or more unlabeled molecules, 50,000 or more unlabeled molecules, 100,000 or more unlabeled molecules, 500,000 or more unlabeled molecules, or 1×10⁶ or more unlabeled molecules. In some embodiments, the plurality of unlabeled molecules comprises 1×10⁶ or more unlabeled molecules.

In some embodiments, each respective instance in the subset of instances is a corresponding graph comprising a corresponding plurality of nodes and a corresponding plurality of edges, wherein each respective node in the corresponding plurality of nodes is a synthon used in the respective instance, and each edge in the corresponding plurality of edges is between a first node and a second node in the corresponding plurality of nodes and is associated with at least a conversion efficiency in the respective instance between the first node and the second node.

In some embodiments, the model is a deep neural network, and the model further generates, as output, an uncertainty estimation for the improved conversion value for the first molecular reaction. Additional examples of deep neural networks generating, as output, an uncertainty estimation are disclosed in, for example, Lakshminarayanana et al., “Simple and Scalable Predictive Uncertainty Estimation using Deep Ensembles,” arXiv 2017, which is hereby incorporated by reference in its entirety.

In some embodiments, the uncertainty estimation may be informed by performing the optimization of the molecular reaction at least once, at least twice, at least three times, or at least three times or more, each with a plurality of instances of the first molecular reaction using a first plurality of at least 4 synthons and an original plurality of normalized conditions. In some embodiments, increasing the number of times the optimization is performed provides improved conversion values and reduces the value of the uncertainty estimates. In some embodiments such repetition allows for resolving aleatory uncertainty and/or epistemic contributions to the uncertainty estimates. See, for example, Fox and Ülkümen, “Distinguishing Two Dimensions of Uncertainty (2011)”, in Essays in Judgment and Decision Making, Brun, W., Kirkebøen, G. and Montgomery, H., eds. Oslo: Universitetsforlaget., Available at SSRN: https://ssrn.com/abstract=3695311 or http://dx.doi.org/10.2139/ssrn.3695311, which is hereby incorporated by reference.

In some embodiments, the model is not a deep neural network.

In some embodiments, a reinforcement learning policy is used in which the model is used as an oracle for the reinforcement learning policy. Additional examples of reinforcement learning policies are disclosed in, for example, Shulamn et al., “Proximal Policy Optimization Algorithms,” arXiv 2017, which is hereby incorporated by reference in its entirety.

In some embodiments, the amount of each respective synthon in the plurality of synthons used in each respective instance of the molecular reaction is in a reaction amount range. In some embodiments, the reaction amount range is a first reaction amount range.

In some embodiments, the reaction amount range is between 0.0001 millimoles and 1 mole of the respective synthon. In some embodiments, the reaction amount range is between 0.001 millimoles and 1 mole of the respective synthon. In some embodiments, the reaction amount range is between 0.001 millimoles and 0.001 moles of the respective synthon. In some embodiments, the reaction amount range is between 0.001 moles and 1 mole of the respective synthon. In some embodiments, the reaction amount range is between 0.0005 millimoles and 0.005 millimoles of the respective synthon. In some embodiments, the reaction amount range is between 0.002 millimoles and 1.5 millimoles of the respective synthon. In some embodiments, the reaction amount range is between 0.01 moles and 0.5 moles of the respective synthon. In some embodiments, the reaction amount range is between 0.0001 millimoles and 0.001 millimoles, 0.001 millimoles and 2 millimoles, 0.01 moles and 0.1 moles, 0.05 moles and 0.1 moles, or 0.01 moles and 0.05 moles of the respective synthon.

In some embodiments, the reaction amount range is between 50 g/mol and 1000 g/mol of the respective synthon. In some embodiments, the reaction amount range is between 150 g/mol and 300 g/mol of the respective synthon. In some embodiments, the reaction amount range is between 100 g/mol and 500 g/mol, 100 g/mol and 400 g/mol, or 125 g/mol and 350 g/mol of the respective synthon.

In some embodiments, the reaction amount range is between 0.001 grams and 1 gram of the respective synthon. In some embodiments, the reaction amount range is between 0.01 grams and 0.5 grams of the respective synthon. In some embodiments, the reaction amount range is between 0.01 grams and 0.1 grams, 0.05 grams and 0.1 grams, or 0.01 grams and 0.05 grams of the respective synthon.

In some embodiments, an absolute volume of each instance of the molecular reaction in the plurality of instances of the molecular reaction is in a reaction volume range.

In some embodiments, the reaction volume range is between 1 microliter and 10,000 microliters. In some embodiments, the reaction volume range is between 10 microliters and 1800 microliters. In some embodiments, the reaction volume range is between 50 microliters and 5000 microliters. In some embodiments, the reaction amount range is between 5 microliters and 500 microliters, 100 microliters and 1000 microliters, 100 microliters and 500 microliters, or 500 microliters and 1000 microliters.

In some embodiments, each edge in the corresponding plurality of edges is further associated with any combination of a solvent, a concentration, a temperature, a reaction volume, an incubation time, a stoichiometry of synthons, or a stoichiometry of reagents. In other words, in some embodiments, the affect of solvent, synthon concentration, reaction temperature, reaction volume, incubation time, synthon stoichiometry, and/or reagent stoichiometry on reaction efficiency is tracked in some embodiments.

In some embodiments, the method further includes using the subset of instances 134 to adjust one or more parameters 162 in a plurality of parameters of the model 160 (e.g., a reinforcement learning model), thereby obtaining an updated plurality of parameters for the model.

In some embodiments, the model is adjusted (e.g., rewarded) in response to output normalized conditions that produce compounds having at least the threshold conversion value. Alternatively or additionally, in some embodiments, the model is adjusted (e.g., penalized) in response to output normalized conditions that produce compounds that fail to have at least the threshold conversion value.

In some embodiments, the model comprises a plurality of at least 1000 parameters, and the using further comprises applying a respective difference to a loss function to obtain a respective output of the loss function, where the respective difference is between, for each respective instance in the subset of instances, (a) the respective conversion value of the respective instance and (b) a threshold conversion value for the respective conversion value of the respective instance. In some embodiments, the using further comprises using the respective output of the loss function to adjust the one or more parameters in the plurality of parameters.

As described above, in some embodiments, for each respective instance in the plurality of instances of the molecular reaction, the output of the model can be used to select a corresponding set of normalized conditions for use in performing the molecular reaction. For instance, for each respective instance in the plurality of instances, the outcome of the molecular reaction is a generated compound, for which a conversion value is determined. Thus, the conversion value of the compound produced by the predicted (outputted) normalized conditions for a respective instance serves as a predicted label, while the threshold conversion value serves as an actual or measured label. Moreover, those normalized reaction conditions (input to the model) that lead to compounds with high predicted conversion values can be selected for additional use with additional synthons in accordance with the molecular reaction, whereas those normalized reaction conditions (input to the model) that lead to compounds with low predicted conversion values can be avoided in future experiments.

In some embodiments, the model takes as input the reaction (e.g. the identity of at least 4 synthons and a plurality of normalized conditions) and outputs a calculated conversion value, and accordingly the model can be used to search for updated normalization conditions by inputting into the model various normalized conditions and synthons until the model identifies one such combination as having a desired and/or improved conversion value (e.g. greater than 80%, greater than 90%, greater than 95%, greater than 99%).

In some embodiments the model supports inverse modeling. For example, in some embodiments the model is a generative adversarial network or a variation autoencoder. Such a model, once trained, can be used in the inverse modeling context. In this context, rather than inputting into the model synthons and reaction conditions and obtaining as output a predicted conversion efficiency of the synthesized molecule, optionally with a measure of uncertainty in this conversion efficiency, the model input and outputs is reversed such that a given molecule is to be synthesized by the first modeling reaction, e.g., with a desired in compound efficiency, is inputted into the layer of the model that was normally used as model output, and the model provides suggested normalized reaction conditions and/or synthons to be used in accordance with the first reaction at the layer of the model that was normally used to input such data during model training.

In some addition embodiments the model supports inverse modeling. For example, in some embodiments the model is a generative adversarial network or a variation autoencoder. Such a model, once trained, can be used in the inverse modeling context. In this context, rather than inputting into the model synthons and reaction conditions and obtaining as output a predicted conversion efficiency, the model input and outputs is reversed such that a desired compound efficiency is inputted into the layer of the model that was normally used as model output, and the model provides suggested normalized reaction conditions that support the desired compound efficiency, in accordance with the first reaction, at the layer of the model that was normally used to input such data during model training. In some embodiments errors in the predicted labels (e.g., errors in the conversion value corresponding to the normalized conditions produced by the model), as verified against the actual labels, are then back-propagated through the parameters of the model (e.g., a reinforcement learning model) in order to train the model. In an example embodiment, a model of the present disclosure is trained against the errors in the predicted labels made by the model, in view of the actual labels, by stochastic gradient descent. In some embodiments, model training involves modifying the parameters of one or more models, or any components or ensembles thereof. In some embodiments, the parameters are further constrained with various forms of regularization such as L1, L2, weight decay, and dropout.

In an exemplary embodiment, the model is trained against the errors in the model prediction (e.g., error in the conversion efficiency) made by the model 120, in view of the actual conversion values that is known for instance of the molecular reaction, using a loss function such as cross-entropy loss (Hastie et al., 2009, The elements of statistical learning: data mining, inference, and prediction Vol. 2, pp. 1-758), New York, Springer, which is hereby incorporated by reference) or focal loss (Lin et al., 2017, “Focal loss for dense object detection,” In Proceedings of the IEEE international conference on computer vision, pp. 2980-2988, which is hereby incorporated by reference). In an exemplary embodiment, the model is trained against the errors in the model prediction (e.g., error in the conversion value) made by the model, in view of the actual conversion value known for each reaction, by stochastic gradient descent with the AdaDelta adaptive learning method (Zeiler, 2012 “ADADELTA: an adaptive learning rate method,” CoRR, vol. abs/1212.5701, which is hereby incorporated by reference), and the back propagation algorithm provided in Rumelhart et al., 1988, “Neurocomputing: Foundations of research,” ch. Learning Representations by Back-propagating Errors, pp. 696-699, Cambridge, MA, USA: MIT Press, which is hereby incorporated by reference. Such a refinement technique is just one of many examples for training a model, each of which is within the scope of the present disclosure.

In some embodiments, the plurality of parameters includes at least 10, at least 100, at least 1000, at least 10,000, at least 100,000, at least 1×10⁶, at least 1×10⁷, or more parameters. In some embodiments, the plurality of parameters comprises 500,000 or more parameters, or 1×10⁶ or more parameters. In some embodiments, the plurality of parameters includes no more than 1×10⁸, no more than 1×10⁷, no more than 1×10⁶, no more than 100,000, no more than 10,000, no more than 1000, or no more than 100 parameters. In some embodiments, the plurality of parameters consists of from 10 to 1000, from 100 to 100,000, from 10,000 to 1×10⁷, or from 1×10⁶ to 1×10⁸ parameters. In some embodiments, the plurality of parameters falls within another range starting no lower than 10 parameters and ending no higher than 1×10⁸ parameters.

In some embodiments, the method further includes producing, subsequent to obtaining the updated plurality of parameters 162, as output from the model 160 (e.g., a reinforcement learning model), responsive to inputting the plurality of synthons 122 into the model with the updated plurality of parameters, an updated plurality of normalized conditions 142 for the molecular reaction 132.

In some embodiments, the method further includes repeating the performing b), obtaining c), selecting d), using e), and producing f) until the respective conversion value for each respective instance in the plurality of instances of the molecular reaction satisfies a first threshold conversion value criterion. In other words, in some embodiments, the model is iteratively trained (e.g., adjusted) until it satisfactorily outputs normalized conditions that produce compounds having conversion values above a threshold conversion value.

In some embodiments, the training is repeated for a plurality of training iterations. In some embodiments, the plurality of training iterations comprises at least 10, at least 100, at least 1000, at least 10,000, at least 100,000, or at least 1×10⁶ training iterations. In some embodiments, the plurality of training iterations includes no more than 1×10⁷, no more than 1×10⁶, no more than 100,000, no more than 10,000, no more than 1000, or no more than 100 training iterations. In some embodiments, the plurality of training iterations consists of from 10 to 1000, from 100 to 100,000, from 10,000 to 1×10⁶, or from 1×10⁶ to 1×10⁷ training iterations. In some embodiments, the plurality of training iterations falls within another range starting no lower than 10 training iterations and ending no higher than 1×10⁷ training iterations.

Accordingly, in some embodiments, the molecular reaction is selected from a plurality of molecular reactions and the method further comprises, for each respective molecular reaction in the plurality of molecular reactions, obtaining a different respective model in a plurality of models. In some implementations, the selecting a), performing b), obtaining c), selecting d), and using e) is repeated for each respective molecular reaction in the plurality of molecular reactions, thereby obtaining a corresponding updated plurality of parameters for the respective model corresponding to the respective molecular reaction in the plurality of molecular reactions.

In some embodiments, one or more steps of the optimization are repeated to prepare a second subset of instances. In some embodiments, the performing ii), obtaining iii), and selecting iv) are repeated within the optimization.

In some embodiments, the optimization further comprises performing a second plurality of instances of the first molecular reaction, using (a) the first plurality of synthons and the updated plurality of normalized conditions and (b) the automated synthesis platform. In some embodiments, for each respective instance of the first molecular reaction, transforming, with the automated synthesis platform, at least a subset of the plurality of synthons using the first molecular reaction, thereby generating a second plurality of compounds.

In some embodiments, the second set of instances and the corresponding conversion value of each instance of the second subset of instances are used to retrain a first model.

In some embodiments, the first model is retrained by using i) the second subset of instances as independent variables and ii) the corresponding conversion value of each instance of the second subset of instances as dependent variables, to guide adjustment of one or more parameters in the plurality of parameters of the first model, so that the first model produces a calculated conversion value in agreement with the corresponding conversion value of each instance of the second subset of instances upon input of the subset of instances into the first model. For example, in some embodiments, the independent variable is a subset of reaction conditions and/or synthons, wherein each instance is or comprises one or more conditions and/or synthons used in a reaction well and the dependent variable is the conversion value.

In some embodiments, the optimization further comprises using, subsequent to obtaining the updated plurality of parameters, the first model to search for and identify a second plurality of compounds that collectively have an improved conversion value for the first molecular reaction relative to the first plurality of compounds.

In some embodiments, the model is trained in accordance with a loss function, an assent function, or a regression.

In some embodiments, a single model is obtained that is agnostic to molecular reaction type.

In some embodiments, the model is a reinforcement learning model. In some embodiments, the reinforcement learning system comprises four main elements—an agent, a policy, a reward signal, and a value function, where the behavior of the agent is defined in terms of the policy. In some embodiments, the reinforcement learning system comprises a learning algorithm. In some implementations, the learning algorithm is an on-policy learning algorithm or an off-policy learning algorithms. On-policy learning algorithms evaluate and improve the same policy that is being used to select the agent's actions. Off-policy learning algorithms evaluate and improve policies that are different from the policy being used for action selection. Reinforcement learning is further described, for example, in Sutton R S, Barto A G, “Reinforcement learning: an introduction,” IEEE Transactions on Neural Networks. 1998; 9(5):1054-1054, which is hereby incorporated herein by reference in its entirety. In some implementations, the model is any of the model architectures disclosed herein.

In aspects, after retraining the first model, the first model can be searched and used to identify molecular reactions, including associated reaction conditions, with improved conversion values compared to the conversion values prior to retraining of the first model. In some embodiments, the various normalized conditions and synthons are inputted into the first model until the first model identifies a combination of normalized conditions and synthons, that were inputted into the model, by scoring such normalized conditions and synthons, as model output, with a high conversion value and/or an improved conversion value compared to the conversion value prior to retraining of the first model.

Accordingly, in some embodiments, the optimization further includes using, subsequent to obtaining an updated plurality of parameters, the first model to search for and identify an updated plurality of normalized conditions for the first molecular reaction that collectively have an improved conversion value compounds synthesized by the first molecular reaction relative to the original plurality of normalized conditions used to synthesize compounds by the first molecular reaction.

In aspects, the optimization further includes the selection of a second molecular reaction. In some embodiments, the optimization includes steps described herein for a first molecular reaction. In some embodiments, selection of a second molecular reaction permits a convergent synthesis by converting a first plurality of compounds into a second plurality of synthons, which can be used along with a second original plurality of normalized conditions to perform a second plurality of instances of the second molecular reaction and produce a second plurality of compounds.

In some embodiments, a second molecular reaction is selected using the computing system, in which the second molecular reaction is in the multistep synthesis, and in which the computing system informs the automated synthesis platform of the second molecular reaction.

In some embodiments, a subset of the first plurality of compounds is selected based on at least the first threshold conversion value of each respective compound of the first plurality of compounds.

In some embodiments, the subset of the first plurality of compounds comprises at least 2 or more, at least 4 or more, at least 5 or more, at least 10 or more, at least 20 or more, at least 50 or more, at least 100 or more, at least 500 or more, at least 1000 or more, at least 10,000, or at least 100,000 compounds. In some embodiments, the subset of the first plurality of compounds comprises 100 or more, 500 or more, 1000 or more, 2000 or more or 10,000 or more compounds. In some embodiments, the subset of the first plurality of compounds comprises no more than 1×10⁶, no more than 100,000, no more than 10,000, no more than 1000, no more than 100, or no more than 50 compounds. In some embodiments, the subset of the first plurality of compounds consists of from 2 to 20, from 10 to 100, from 50 to 1000, from 500 to 10,000, from 2000 to 500,000, or from 100,000 to 1×10⁶ compounds.

In some embodiments, the respective conversion value is a percent yield of a corresponding compound obtained for the respective instance of the molecular reaction determined as a percent of amount (e.g., mass, volume) of expected product to the actual amount of product produced in the respective instance of the molecular reaction. In some embodiments, the first threshold conversion value is at least about 20%, at least about 30%, at least about 40%, at least 5 about 0%, at least about 60%, at least about 70%, at least about 80%, at least about 85%, at least about 90%, or at least about 95%.

In some embodiments, the first threshold conversion value is at least about 40%, at least about 50%, or at least about 60%. In some embodiments, the first threshold conversion value is at least about 10%, at least about 20%, at least about 30%, at least about 40%, at least about 50%, at least about 60%, at least about 65%, at least about 70%, at least about 75%, at least about 80%, at least about 85%, or at least about 90%. In some embodiments, the first threshold conversion value is no more than about 95%, no more than about 90%, no more than about 80%, no more than about 70%, no more than about 60%, no more than about 50%, no more than about 40%, no more than about 30%, or no more than about 20%. In some embodiments, the first threshold conversion value is from about 10% to about 30%, from about 20% to about 50%, from about 30% to about 60%, from about 40% to about 80%, from about 50% to about 90%, or from about 70% to about 95%. In some embodiments, the first threshold conversion value falls within another range starting no lower than about 10% and ending no higher than about 95%.

In some embodiments, the first plurality of compounds comprises at least 2 or more, at least 4 or more, at least 5 or more, at least 10 or more, at least 20 or more, at least 50 or more, at least 100 or more, at least 500 or more, at least 1000 or more, at least 10,000, or at least 100,000 compounds. In some embodiments, the first plurality of compounds comprises 100 or more, 500 or more, 1000 or more, 2000 or more or 10,000 or more compounds. In some embodiments, the first plurality of compounds comprises no more than 1×10⁶, no more than 100,000, no more than 10,000, no more than 1000, no more than 100, or no more than 50 compounds. In some embodiments, the first plurality of compounds consists of from 2 to 20, from 10 to 100, from 50 to 1000, from 500 to 10,000, from 2000 to 500,000, or from 100,000 to 1×10⁶ compounds.

In some embodiments, each compound in the first plurality of compounds satisfies two or more rules, three or more rules, or all four rules of the Lipinski's rule of Five: (i) not more than five hydrogen bond donors, (ii) not more than ten hydrogen bond acceptors, (iii) a molecular weight under 500 Daltons, and (iv) a Log P under 5.

In some embodiments, each compound in the first plurality of compounds is an organic compound having a molecular weight of less than 500 Daltons, less than 1000 Daltons, less than 2000 Daltons, less than 4000 Daltons, less than 6000 Daltons, less than 8000 Daltons, less than 10000 Daltons, or less than 20000 Daltons. In some embodiments, each compound in the first plurality of compounds is an organic compound having a molecular weight of between 400 Daltons and 10000 Daltons.

In some embodiments, the optimization further comprises performing a second plurality of instances of the second molecular reaction using the second plurality of synthons and a second original plurality of normalized conditions using the automated synthesis platform. In some embodiments, for each respective instance of the second molecular reaction, a transformation, with the automated synthesis platform, of at least a subset of the second plurality of synthons is done using the second molecular reaction, thereby generating a second plurality of compounds. For example, through the use of the automated synthesis platform, additional compounds (e.g. the second plurality of compounds) can be prepared using a second molecular reaction from synthons generated from compounds prepared using a first molecular reaction (e.g. a second plurality of synthons) and normalized conditions (e.g. a second original plurality of normalized conditions).

In some embodiments, the optimization further comprises selecting a second subset of instances from the second plurality of instances based on at least a second threshold conversion value for the second respective conversion value of each respective instance, where the automated synthesis platform informs the computing system of the second subset of instances.

In some embodiments, the optimization further comprises obtaining, for each respective instance of the second molecular reaction, a second respective conversion value for the respective instance.

In some embodiments, the second subset of instances comprises at least 10, at least 50, at least 100, at least 500, at least 1000, at least 10,000, at least 100,000, at least 1×10⁶, at least 1×10⁷, at least 1×10⁸, or at least 1×10⁹ instances. In some embodiments, the second subset of instances comprises no more than 1×10¹⁰, no more than 1×10⁹, no more than 1×10⁸, no more than 1×10⁷, no more than 1×10⁶, no more than 100,000, or no more than 10,000 instances. In some embodiments, the second subset of instances consists of from 1000 to 100,000, from 10,000 to 1×10⁶, from 1×10⁶ to 1×10⁸, or from 1×10⁸ to 1×10¹⁰ instances. In some embodiments, the second subset of instances falls within another range starting no lower than 10 instances and ending no higher than 1×10¹⁰ instances.

In some embodiments, the respective conversion value is a percent yield of a corresponding compound obtained for the respective instance of the molecular reaction determined as a percent of amount (e.g., mass, volume) of expected product to the actual amount of product produced in the respective instance of the molecular reaction. In some embodiments, the second threshold conversion value is at least about 20%, at least about 30%, at least about 40%, at least 5 about 0%, at least about 60%, at least about 70%, at least about 80%, at least about 85%, at least about 90%, or at least about 95%.

In some embodiments, the second threshold conversion value is at least about 40%, at least about 50%, or at least about 60%. In some embodiments, the second threshold conversion value is at least about 10%, at least about 20%, at least about 30%, at least about 40%, at least about 50%, at least about 60%, at least about 65%, at least about 70%, at least about 75%, at least about 80%, at least about 85%, or at least about 90%. In some embodiments, the second threshold conversion value is no more than about 95%, no more than about 90%, no more than about 80%, no more than about 70%, no more than about 60%, no more than about 50%, no more than about 40%, no more than about 30%, or no more than about 20%. In some embodiments, the second threshold conversion value is from about 10% to about 30%, from about 20% to about 50%, from about 30% to about 60%, from about 40% to about 80%, from about 50% to about 90%, or from about 70% to about 95%. In some embodiments, the second threshold conversion value falls within another range starting no lower than about 10% and ending no higher than about 95%.

In some embodiments, the second plurality of compounds comprises at least 2 or more, at least 4 or more, at least 5 or more, at least 10 or more, at least 20 or more, at least 50 or more, at least 100 or more, at least 500 or more, at least 1000 or more, at least 10,000, or at least 100,000 compounds. In some embodiments, the second plurality of compounds comprises 100 or more, 500 or more, 1000 or more, 2000 or more or 10,000 or more compounds. In some embodiments, the second plurality of compounds comprises no more than 1×10⁶, no more than 100,000, no more than 10,000, no more than 1000, no more than 100, or no more than 50 compounds. In some embodiments, the second plurality of compounds consists of from 2 to 20, from 10 to 100, from 50 to 1000, from 500 to 10,000, from 2000 to 500,000, or from 100,000 to 1×10⁶ compounds.

In some embodiments, each compound in the second plurality of compounds satisfies two or more rules, three or more rules, or all four rules of the Lipinski's rule of Five: (i) not more than five hydrogen bond donors, (ii) not more than ten hydrogen bond acceptors, (iii) a molecular weight under 500 Daltons, and (iv) a Log P under 5.

In some embodiments, each compound in the second plurality of compounds is an organic compound having a molecular weight of less than 500 Daltons, less than 1000 Daltons, less than 2000 Daltons, less than 4000 Daltons, less than 6000 Daltons, less than 8000 Daltons, less than 10000 Daltons, or less than 20000 Daltons. In some embodiments, each compound in the second plurality of compounds is an organic compound having a molecular weight of between 400 Daltons and 10000 Daltons.

In some embodiments, the second plurality of instances comprises at least 100, at least 500, at least 1000, at least 10,000, at least 100,000, at least 1×10⁶, at least 1×10⁷, at least 1×10⁸, or at least 1×10⁹ instances. In some embodiments, the second plurality of instances comprises no more than 1×10¹⁰, no more than 1×10⁹, no more than 1×10⁸, no more than 1×10⁷, no more than 1×10⁶, no more than 100,000, or no more than 10,000 instances. In some embodiments, the second subset of instances consists of from 1000 to 100,000, from 10,000 to 1×10⁶, from 1×10⁶ to 1×10⁸, or from 1×10⁸ to 1×10¹⁰ instances. In some embodiments, the second plurality of instances falls within another range starting no lower than 1000 instances and ending no higher than 1×10¹⁰ instances.

In some embodiments, the optimization further comprises training a second model by using using i) the second subset of instances as independent variables and ii) the corresponding conversion value of each instance of the second subset of instances as dependent variables, to guide adjustment of one or more parameters in a plurality of parameters of the second model, so that the second model produces a calculated conversion value in agreement with the corresponding conversion value of each instance of the second subset of instances upon input of the second subset of instances into the second model. For example, in some embodiments, the independent variable is a subset of reaction conditions and/or synthons, wherein each instance is a reaction well. In some embodiments, the output includes the identity of the compound.

In some embodiments, the first model and the second model are the same model. In some embodiments, the first model and the second model are different models.

In some embodiments, the transforming, with the automated synthesis platform, further comprises reacting each respective synthon of the second subset of the second plurality of synthons with a synthon of a third plurality of synthons.

In some embodiments, each respective normalized condition in the first plurality of normalized conditions and/or the second plurality of normalized conditions is selected from the group consisting of: synthon type, reagents, solvents, concentrations, order of addition, amount of equivalents for addition, synthon scope, temperature, incubation time, stoichiometry of synthons, and stoichiometry of reagents. See, e.g., above section titled “Compound Synthesis.”

### Selecting Synthons.

In some embodiments, the apparatus 10 is useful for selecting synthons for a molecular reaction. In some embodiments, the apparatus comprises an automated synthesis platform (e.g. automated synthesis platform 200) and a computing system (e.g. computing system 100). In some embodiments, the computing system comprises one or more processors and memory addressable by the one or more processors, the memory storing the model. In some embodiments, the computing system 100 informs the automated synthesis platform 200 of the molecular reaction (see, for example, FIG. 1). In some embodiments, the molecular reaction is a multistep reaction.

In some embodiments, the automating further includes performing a first plurality of instances 134 of the molecular reaction 132 using a plurality of synthons 122 (e.g., at least 4 synthons) and a plurality of normalized conditions 142, comprising, for each respective instance 134 of the first molecular reaction 132, transforming, with the automated synthesis platform (e.g. automated synthesis platform 200) at least a subset of the plurality of synthons 122 using the molecular reaction, thereby generating a first plurality of compounds 152. For example, in some such embodiments, the first plurality of compounds is generated over the plurality of instances of the first molecular reaction. In some embodiments, each respective instance of the first molecular reaction generates a compound. In some embodiments, each respective instance of the first molecular reaction generates a first plurality of compounds.

In some embodiments, the automating further includes obtaining, for each respective instance 134 of the molecular reaction 132, a respective conversion value 154 for the respective instance.

In some embodiments, the automating further includes selecting a first subset of instances from the plurality of instances 134 based on at least a first threshold conversion value 156 for the respective conversion value 154 of each respective instance. In some embodiments, the automated synthesis platform (e.g., automated synthesis platform 200) informs the computing system of the first subset of instances.

In some embodiments, the automating further includes selecting a first subset of synthons 158 that are enriched in the first subset of instances relative to the plurality of instances of the molecular reaction.

### Generating Worklists.

In some embodiments, the optimization and/or automating further includes performing a test instance of the molecular reaction using a test plurality of synthons, comprising (i) obtaining, responsive to inputting at least the test plurality of synthons into the model, a corresponding test set of normalized conditions as respective output from the model, and (ii) transforming a corresponding subset of synthons in the test plurality of synthons under the corresponding test set of normalized conditions using the molecular reaction, thereby generating a respective test compound. In some embodiments, the method further includes obtaining a respective conversion value for the test instance of the molecular reaction.

In some embodiments, the test plurality of synthons comprises a first subplurality of synthons and a second subplurality of synthons, the first subplurality of synthons comprises at least 4 synthons of a first reactant type, and the second subplurality of synthons comprises at least 6 synthons of a second reactant type.

As described above, in some embodiments, a respective subplurality of synthons in the plurality of synthons comprises at least 2, at least 4, at least 5, at least 10, at least 20, at least 50, at least 100, or at least 500 synthons. In some embodiments, a respective subplurality of synthons in the plurality of synthons comprises no more than 1000, no more than 500, no more than 100, no more than 50, no more than 20, or no more than 10 synthons. In some embodiments, a respective subplurality of synthons in the plurality of synthons consists of from 2 to 10, from 4 to 30, from 20 to 100, from 80 to 500, or from 300 to 1000 synthons. In some embodiments, a respective subplurality of synthons in the plurality of synthons falls within another range starting no lower than 2 synthons and ending no higher than 1000 synthons.

In some embodiments, the respective conversion value for the test instance is determined as a percent yield of a corresponding compound obtained for the test instance of the molecular reaction.

In some embodiments, the optimization and/or automating further includes (iii) evaluating a performance of the test instance of the molecular reaction based upon a comparison of the respective conversion value with the threshold conversion value. In some implementations, when the performance of the test instance satisfies a second threshold conversion value criterion, the corresponding test set of normalized conditions is assigned as reaction conditions in a compound synthesis pipeline, and when the performance of the test instance fails to satisfy the second threshold conversion value criterion, the obtaining (i), transforming (ii), and evaluating (iii) is repeated. In some embodiments, the repeating the obtaining (i), transforming (ii), and evaluating (iii) is performed until a satisfactory test instance is achieved.

In some embodiments, the assigning the corresponding test set of normalized conditions as reaction conditions in a compound synthesis pipeline further comprises generating a worklist for automated synthesis of a corresponding compound obtained for the test instance of the molecular reaction.

Automated devices, including automated synthesis devices, are described in greater detail elsewhere herein (see, for instance, the section entitled “Compound synthesis,” above).

### Selecting Molecular Reactions.

In some embodiments, the optimization and/or automating further includes, prior to the selecting a): obtaining a first candidate molecule from a plurality of candidate molecules; determining, for the first candidate molecule, a corresponding one or more molecular reactions in a plurality of molecular reactions; and selecting the molecular reaction from the corresponding one or more molecular reactions for the first candidate molecule.

Generally, in some such embodiments, the molecular reaction is selected from the one or more molecular reactions for a first candidate molecule. In other words, molecular reactions to be optimized are selected in order to produce candidate molecules of interest. In some implementations, candidate molecules of interest are selected based on their target properties, including but not limited to drug-likeness, binding score, selectivity score, and/or ADME score. Thus, a candidate molecule that has properties of interest is, in some embodiments, selected for optimization of synthesis, screening, and/or validation. In some embodiments, as described above, the synthesis, screening, and/or validation is performed in an automated fashion. Moreover, in some embodiments, the optimization of such synthesis, screening, validation, and/or synthons, conditions, or protocols for performing the same, is performed in an automated fashion.

In an example embodiment, molecular reactions are chosen primarily based on their utilization in candidate molecule selections. The most commonly utilized reactions that are used to enumerate compounds that are selected for synthesis are prioritized for development to ensure the reactions available on the automated synthesis platform are of importance for the types of compounds required for target pipeline programs. Additionally, in some embodiments, molecular reactions which provide similar bond connections, but which utilize different reagents or conditions are added for development to ensure there are multiple ways to make any respective target candidate molecule.

In some embodiments, the optimization and/or automating further includes obtaining the plurality of candidate molecules by a procedure comprising: i) obtaining the plurality of molecular reactions and a plurality of initial synthons; ii) obtaining, for each respective initial synthon in the plurality of initial synthons, a respective transformation of the respective initial synthon that represents a corresponding one or more molecular reactions in the plurality of molecular reactions, thereby generating a plurality of intermediate synthons; iii) removing, from the plurality of intermediate synthons, one or more respective intermediate synthons based on a respective first score for an interaction between each respective intermediate synthon in the plurality of intermediate synthons and a target entity; iv) assigning, after the removing, the plurality of intermediate synthons to the plurality of initial synthons; and v) repeating the obtaining ii), removing iii), and assigning iv) until a respective second score for the interaction between each respective intermediate synthon in the plurality of intermediate synthons and the target entity satisfies a threshold exit criterion, thereby generating the plurality of candidate molecules.

In some embodiments, the plurality of initial synthons comprises at least 1×10⁶ initial synthons. In some embodiments, the plurality of initial synthons comprises at least 1000, at least 5000, at least 10,000, at least 100,000, at least 1×10⁶, at least 1×10⁷, at least 1×10⁸, at least 1×10⁹, at least 1×10¹⁰, at least 1×10¹¹, or at least 5×10¹¹ initial synthons. In some embodiments, the plurality of initial synthons comprises no more than 1×10¹², no more than 1×10¹¹, no more than 1×10¹⁰, no more than 1×10⁹, no more than 1×10⁸, no more than 1×10⁷, no more than 1×10⁶, no more than 100,000, or no more than 10,000 initial synthons. In some embodiments, the plurality of initial synthons consists of from 1000 to 100,000, from 10,000 to 1×10⁷, from 1×10⁶ to 1×10⁸, from 1×10⁸ to 1×10¹¹, or from 1×10⁹ to 1×10¹² initial synthons. In some embodiments, the plurality of initial synthons falls within another range starting no lower than 1000 initial synthons and ending no higher than 1×10¹² initial synthons.

In some embodiments, the plurality of candidate molecules comprises at least 1×10⁶ candidate molecules. In some embodiments, the plurality of candidate molecules comprises at least 1000, at least 5000, at least 10,000, at least 100,000, at least 1×10⁶, at least 1×10⁷, at least 1×10⁸, at least 1×10⁹, at least 1×10¹⁰, at least 1×10¹¹, or at least 5×10¹¹ candidate molecules. In some embodiments, the plurality of candidate molecules comprises no more than 1×10¹², no more than 1×10¹¹, no more than 1×10¹⁰, no more than 1×10⁹, no more than 1×10⁸, no more than 1×10⁷, no more than 1×10⁶, no more than 100,000, or no more than 10,000 candidate molecules. In some embodiments, the plurality of candidate molecules consists of from 1000 to 100,000, from 10,000 to 1×10⁷, from 1×10⁶ to 1×10⁸, from 1×10⁸ to 1×10¹¹, or from 1×10⁹ to 1×10¹² candidate molecules. In some embodiments, the plurality of candidate molecules falls within another range starting no lower than 1000 candidate molecules and ending no higher than 1×10¹² candidate molecules.

In some embodiments, the target entity is a target macromolecule or target macromolecule complex. In some embodiments, the target macromolecule or macromolecule complex comprises one or more active sites to which a respective candidate molecule can bind.

In some embodiments, each respective candidate molecule is a chemical compound. In some embodiments, each respective candidate molecule is a ligand and/or a substrate. In some embodiments, a respective candidate molecule is a large polymer or macromolecule, such as an antibody. In some embodiments, a respective candidate molecule is an organic or inorganic compound.

In some embodiments, a respective candidate molecule satisfies two or more rules, three or more rules, or all four rules of the Lipinski's rule of Five: (i) not more than five hydrogen bond donors, (ii) not more than ten hydrogen bond acceptors, (iii) a molecular weight under 500 Daltons, and (iv) a Log P under 5. See, Lipinski, 1997, Adv. Drug Del. Rev. 23, 3, which is hereby incorporated herein by reference in its entirety. In some embodiments, the respective candidate molecule satisfies one or more criteria in addition to Lipinski's Rule of Five. For example, in some embodiments, the respective candidate molecule has five or fewer aromatic rings, four or fewer aromatic rings, three or fewer aromatic rings, or two or fewer aromatic rings.

In some embodiments, a respective candidate molecule has a molecular weight of at least 100, at least 500, at least 1000, at least 2000, at least 5000, or at least 10,000 Daltons. In some embodiments, a respective candidate molecule has a molecular weight of no more than 20,000, no more than 10,000, no more than 8000, no more than 6000, no more than 4000, no more than 2000, no more than 1000, or no more than 500 Daltons. In some embodiments, a respective candidate molecule has a molecular weight of from 100 to 500, from 500 to 2000, from 1000 to 8000, or from 5000 to 20,000 Daltons. In some embodiments, a respective candidate molecule has a molecular weight that falls within another range starting no lower than 100 Daltons and ending no higher than 20,000 Daltons. However, some embodiments of the disclosed systems and methods have no limitation on the size of the candidate molecule. In some embodiments, the molecular weight is represented in g/mol by converting Daltons into g/mol (1 Dalton=1 g/mol).

In some embodiments, for each respective intermediate synthon in the plurality of intermediate synthons: the respective first score is obtained using at least a corresponding first plurality of interaction features for a complex formed between the respective intermediate synthon and the target entity, and the respective second score is obtained using at least a corresponding second plurality of interaction features for a complex formed between the respective molecular intermediate and the target macromolecule or target macromolecule complex.

In some embodiments, a respective score for a respective molecule characterizes or otherwise indicates an interaction between the respective molecule and a target (or off-target) macromolecule or macromolecule complex. In some implementations, a respective score is a causal interaction feature score that is obtained using one or more interaction features associated with a conformation of the respective molecule when complexed to the target (or off-target) macromolecule or macromolecule complex. However, any suitable method for obtaining interaction scores is contemplated for use in the present disclosure, as will be apparent to one skilled in the art.

In some implementations, a respective score for a respective molecule is based at least on a count of interaction features for a conformation of the respective molecule when complexed to the target (or off-target) macromolecule or macromolecule complex. A count of interaction features can refer to a tally of the plurality of interaction features associated with the respective molecule, but can also refer to any weighted count or computation of causality over the plurality of interaction features.

Accordingly, in some implementations, a respective score is an absolute count, a weighted count, an individual treatment score (e.g., a dot product between an interaction feature vector and corresponding average treatment effects for each respective interaction feature in the interaction feature vector), a weighted individual treatment score, an efficiency score (e.g., a ratio of the number of interaction features for the respective molecule and the number of heavy atoms in the respective molecule), a weighted efficiency score, a diversity score (e.g., a measure of a diversity of interaction feature classes in a plurality of interaction features associated with the respective molecule when complexed to the macromolecule or macromolecule complex), and/or a weighted diversity score.

In some implementations, a weighted score gives greater import to one or more interaction features in a corresponding plurality of interaction features for a respective molecule, compared to other interaction features in the corresponding plurality of interaction features. In an example implementation, a weighted score gives greater weight to a first interaction feature that is selected as or known to be highly causal or associated with a particular property relevant to interaction (e.g., binding potency, selectivity, ADME properties, toxicity, etc.). In such an example implementations, the weighted score gives lesser weight to a second interaction feature that is selected as or known to be a covariate, confounder, or otherwise have lower causality for the particular property.

In some implementations, a weighted score is differentially weighted based on the presence or absence of one or more interaction features in a corresponding plurality of interaction features for a respective molecule. For instance, in some such implementations, a respective score for a respective molecule is predictive of binding when one or more interaction features, or classes thereof, in a first subset of interaction features is present in the corresponding plurality of interaction features for the respective molecule, and is not predictive of binding when none of the interaction features, or classes thereof, in the first subset of interaction features is present in the corresponding plurality of interaction features for the respective molecule. In other words, in some such implementations, a weighted score accounts for interaction features or feature classes that are selected as or known to be essential for a particular interaction property. Alternatively or additionally, in some implementations, a weighted score accounts for interaction features or feature classes that are selected as or known to be adverse or inhibitive to the particular interaction property. In some embodiments, a weighted score is determined by adjusting a corresponding attribute for each respective interaction feature by a weighting factor (e.g., 0.8, 0.2).

In some embodiments, the method further includes filtering the plurality of candidate molecules using, for each respective candidate molecule in the plurality of candidate molecules, one or more interaction features for a complex formed between the respective candidate molecule and the target entity.

In some embodiments, a respective interaction feature is selected from the group consisting of: three-dimensional partial charges, three-dimensional pharmacophores, or molecular dynamics residue interaction time. In some embodiments, a respective interaction feature is selected from the group consisting of hydrophobic interaction, hydrophobic areas, aromatic ring members, hydrogen bond acceptors, hydrogen bond donors, hydrogen bond acceptor in an aromatic ring, negatively charged species, positively charged species, metal coordination, and/or halogen bonds. In some embodiments, a respective interaction feature is a pharmacophore, such as a three-dimensional pharmacophore.

In some embodiments, a respective interaction feature includes one or more corresponding geometric representations and/or one or more attribute values. In some embodiments, the dimensionality and nature of the geometric representations and/or attribute values of interaction features are dependent on the type of interaction feature; that is, a corresponding measurement appropriate for the respective interaction feature, as will be apparent to one skilled in the art. For instance, in some embodiments, a geometric representation of a respective interaction feature is a set of coordinates that indicates the position of the respective interaction feature in three-dimensional space for a respective conformation of the complex formed between a respective molecule and a corresponding target macromolecule or target macromolecule complex. In some embodiments, a geometric representation of a respective interaction feature is a direction vector that indicates the direction or orientation of the respective interaction feature in three-dimensional space for the respective conformation of the complex formed between the respective molecule and the corresponding target macromolecule or target macromolecule complex.

Interaction features are further described, for example, in Jiang L, Rizzo RC, “Pharmacophore-based similarity scoring for dock,” J Phys Chem B. 2015; 119(3):1083-1102; and Arthur G, Oliver W, Klaus B, et al., “Hierarchical graph representation of pharmacophore models,” Front Mol Biosci. 2020; 7:599059, each of which is hereby incorporated herein by reference in its entirety.

### ADDITIONAL EXAMPLE EMBODIMENTS

Another aspect of the present disclosure includes a system, including a memory; one or more processors; and one or more modules stored in the memory and configured for execution by the one or more processors, the one or more modules including instructions for performing any of the methods disclosed above.

Another aspect of the present disclosure includes a non-transitory computer readable storage medium, the non-transitory computer readable storage medium storing one or more programs for execution by one or more processors of a computer system, the one or more computer programs including instructions for performing any of the methods disclosed above.

In some embodiments, the systems and methods disclosed herein are advantageously used in any number of applications, including but not limited to hit discovery, hit-to-lead discovery, lead optimization, off-target side-effect prediction, molecular dynamics simulations, toxicity prediction, potency optimization, selectivity optimization, fitness modeling, drug repurposing, drug resistance prediction, personalized medicine, drug trial design, agrochemical design, and/or materials science.

### EXAMPLES

### Example 1—Improved Molecule Design for Drug Discovery Using Machine Learning Models

Molecular reactions conventionally used in drug discovery are performed by traditional chemistry methods. However, the use of a limited set of molecular reactions has led to a narrowly populated chemical space. In particular, repeated chemical synthesis efforts using similar chemistry and similar molecules does not lead to a greater number of drug candidates; while approximately 100,000,000 molecules have been synthesized in human history, the rate of drug approval has remained relatively constant.

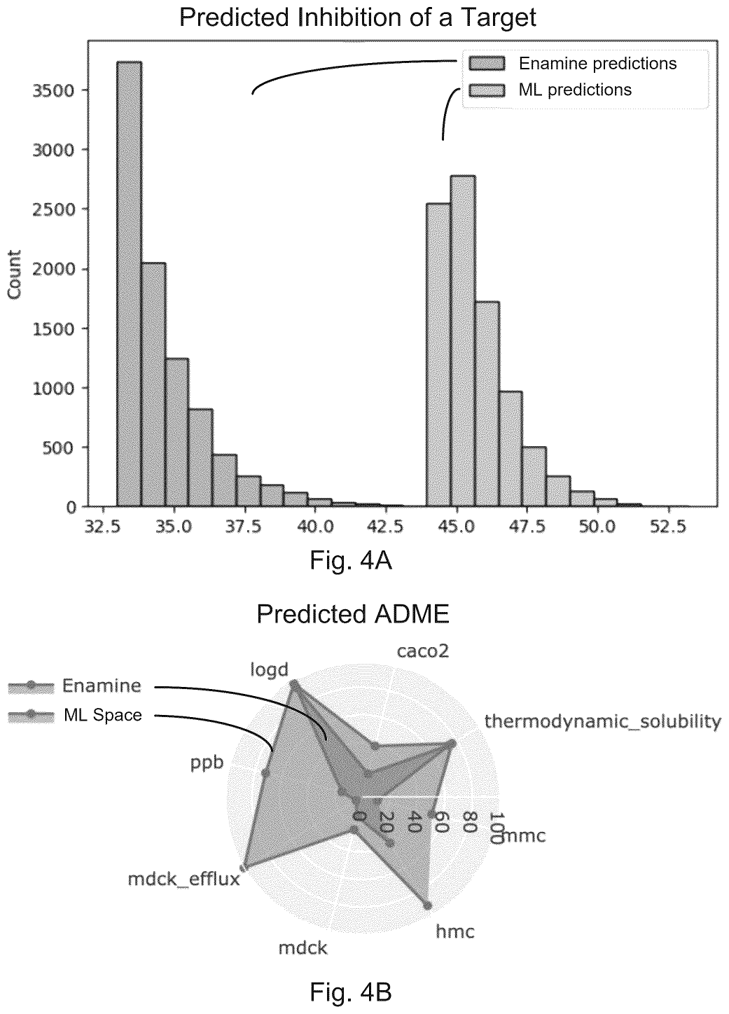

To solve multiparameter problems, such as the discovery of drug-like molecules having properties that will function in vivo, the presently disclosed systems and methods aim to explore new types of molecules in a different chemical space. For instance, FIGS. 4A-B illustrate predicted properties for a set of candidate molecules obtained using machine learning approaches, in accordance with some embodiments of the present disclosure. Compared with Enamine, a widely used, conventional virtual library, the candidate molecules generated using the presently disclosed machine learning approaches were predicted to exhibit higher target inhibition and higher ADME scores.

Automated chemistry has the power to learn new molecular reactions using multiple reaction conditions. Furthermore, the development of new chemistry can lead to novel building blocks and new small molecules for use in the design and development of drug candidates that improve upon traditional methods.

### Buchwald Cross Coupling Reaction

A non-limiting example of a reaction suitable for automated reaction development is the Buchwald cross coupling reaction. Generally, the Buchwald cross coupling reaction is the reaction between an aryl halide and an amine or amide to form a new aryl C—N bond using a palladium catalyst, ligand, and base. Scheme 1 illustrates a non-limiting example of a general synthetic scheme of the Buchwald cross coupling.

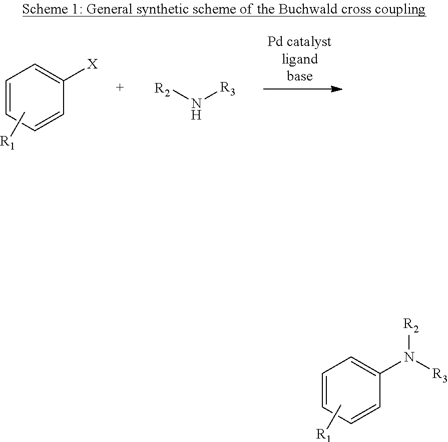

### Reactants

In this Example, for the exploratory and optimization phases of reaction development, six of one reactant and four of another reactant are used to probe the reactivity of desired conditions, and the reactants encompass the reactivity that is being tested (i.e. Buchwald cross coupling). In this case, initial, general reaction conditions for an automated synthesis for the Buchwald cross coupling were examined. The study sought to identify general reaction conditions, including identify a broad variety of reactants capable of carrying out the reaction, and within the 6×4 reagent constraints. The study also included identifying building blocks capable of being identified using liquid chromatography/mass spectroscopy (LCMS) for analysis and determination of percent conversion of the reaction. Desirable building blocks have a molecular weight (MW) of 150 g/mol or greater, are capable of being ionized by electrospray ionization (ESI), and are UV active. Additionally, availability and cost of the reactant are also factor that can be considered in reactant selection.

### Aryl Halides

In this Example, aryl halides are the set of six reactants. Non-limiting examples of factors considered in selecting the aryl halides include the identity of the halide or pseudohalide, the sterics surrounding the halide, and the electronics of the ring. As bromides and chlorides are more common and commercially available than iodides and triflates, two examples of bromides and chlorides were used. Scheme 2 below shows the structures of the six selected aryl halides.

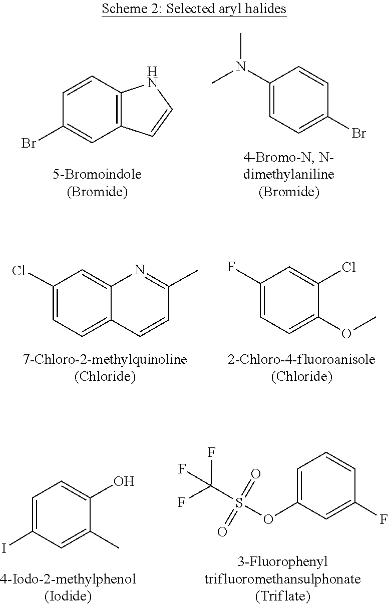

### Amines

In this Example, amines are the set of four reactants. Non-limiting examples of factors considered in selecting the amines include whether the nitrogen is in an amine or an amide, whether the amine is a primary or secondary amine, or whether the amine is an aryl or alkyl substituted amine. The four selected amines are shown in Scheme 3 below. By varying the structures and electronics of the aryl halides and amines, the selected six aryl halides and four amines provide a broad range of reactants for exploring conditions for the automated Buchwald cross coupling.

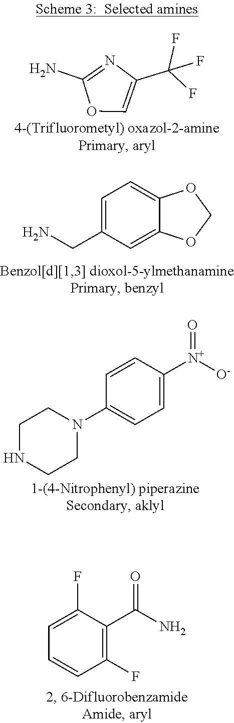 [SMILES: Nc1nc(C(F)(F)F)co1 ; NCc1ccc2c(c1)OCO2 ; O=[N+]([O-])c1ccc(N2CCNCC2)cc1 ; NC(=O)c1c(F)cccc1F]

### Amidation

A non-limiting example of a reaction suitable for automated reaction development is an amidation reaction. Scheme 4 illustrates a non-limiting example of a general synthetic scheme of an amidation reaction.

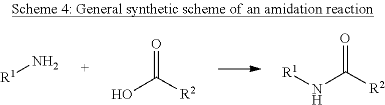

In this Example, six amines and four carboxylic acids are selected as reactants to form a set of 6×4 reactants (see, Schemes 5 and 6 for structures of amines and carboxylic acids). Four different solvents are selected for examination (e.g., N-methyl-2-pyrrolidone (NMP), dimethylformamide (DMF), acetonitrile (MeCN), and dimethyl sulfoxide (DMSO)), thereby providing 96 possible combinations of reactants and solvent for evaluation. The total number of combinations of reactions can be further expanded by treating each of the specific combinations of reactants and solvents with different sets of reagents (e.g., coupling agents, bases, acids, etc.) and under different reaction conditions.

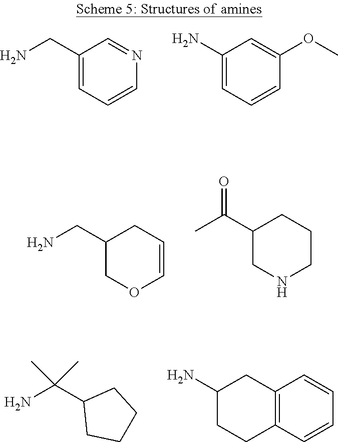

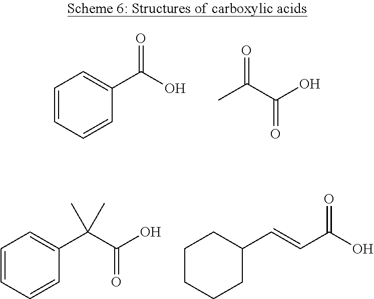

### CONCLUSION

The foregoing description, for purposes of explanation, has been described with reference to specific implementations. However, the illustrative discussions above are not intended to be exhaustive or to limit the implementations to the precise forms disclosed. Many modifications and variations are possible in view of the above teachings. The implementations were chosen and described in order to best explain the principles and their practical applications, to thereby enable others skilled in the art to best utilize the implementations and various implementations with various modifications as are suited to the particular use contemplated.

What is claimed is:

1. An apparatus for improving a first model for use in an optimization of a first molecular reaction, wherein the first molecular reaction is in a multistep synthesis, the apparatus comprising:
a) an automated synthesis platform; and
b) a computing system comprising one or more processors and memory addressable by the one or more processors, the memory storing the first model, wherein the optimization comprises:
i) selecting the first molecular reaction using the computing system, wherein the computing system informs the automated synthesis platform of the first molecular reaction;
ii) performing a first plurality of instances of the first molecular reaction using a first plurality of at least 4 synthons and an original plurality of normalized conditions using the automated synthesis platform, comprising:
for each respective instance of the first molecular reaction, transforming, with the automated synthesis platform, at least a subset of the first plurality of synthons using the first molecular reaction, thereby generating a first plurality of compounds;
iii) obtaining, for each respective instance of the first molecular reaction, a respective conversion value for the respective instance;
iv) selecting a first subset of instances from the first plurality of instances based on at least a first threshold conversion value for the respective conversion value of each respective instance, wherein the automated synthesis platform informs the computing system of the first subset of instances;
v) training the first model by using i) the first subset of instances as independent variables and ii) the corresponding conversion value of each instance of the first subset of instances as dependent variables, to guide adjustment of one or more parameters in a plurality of parameters of the first model, so that the first model produces a calculated conversion value in agreement with the corresponding conversion value of each instance of the first subset of instances upon input of the first subset of instances into the first model; and
vi) using, subsequent to obtaining an updated plurality of parameters, the first model to search for and identify an updated plurality of normalized conditions for the first molecular reaction that collectively have an improved conversion value for the first molecular reaction relative to the original plurality of normalized conditions.

2. The apparatus of claim 1, wherein the first model is a graph neural network.

3. The apparatus of claim 2, wherein the graph neural network is pre-trained, prior to the training v), on a local level using a plurality of unlabeled molecules.

4. The apparatus of claim 3, wherein the plurality of unlabeled molecules is other than the plurality of compounds.

5. The apparatus of claim 3, wherein the plurality of unlabeled molecules is the ZINC15 database.

6. The apparatus of claim 2, wherein each respective instance in the first subset of instances is a corresponding graph comprising a corresponding plurality of nodes and a corresponding plurality of edges, wherein each respective node in the corresponding plurality of nodes is a synthon used in the respective instance, and each edge in the corresponding plurality of edges is between a first node and a second node in the corresponding plurality of nodes and is associated with at least a conversion efficiency in the respective instance between the first node and the second node.

7. The apparatus of claim 1, wherein the using vi) is performed in accordance with a reinforcement learning policy in which the first model is used as an oracle for the reinforcement learning policy.

8. The apparatus of claim 1, wherein an amount of each respective synthon in the first plurality of synthons used in each respective instance of the first molecular reaction is in a first reaction amount range.

9. The apparatus of claim 8, wherein the first reaction amount range is between 0.0005 millimoles and 0.005 millimoles or 0.002 millimoles and 1.5 millimoles of the respective synthon.

10. The apparatus of claim 8, wherein the first reaction amount range is between 150 g/mol and 300 g/mol of the respective synthon.

11. The apparatus of claim 1, wherein an absolute volume of each instance of the first molecular reaction in the plurality of instances of the first molecular reaction is in a first reaction volume range.

12. The apparatus of claim 11, wherein the first reaction volume range is between 10 microliters and 1800 microliters.

13. The apparatus of claim 6, wherein each edge in the corresponding plurality of edges is further associated with any combination of a solvent, a concentrations, a temperature, a reaction volume, an incubation time, a stoichiometry of synthons, or a stoichiometry of reagents.

14. The apparatus of claim 7, wherein the optimization further comprises:
vii) performing a second plurality of instances of the first molecular reaction, using (a) the first plurality of synthons and the updated plurality of normalized conditions and (b) the automated synthesis platform, comprising:
for each respective instance of the first molecular reaction, transforming, with the automated synthesis platform, at least a subset of the plurality of synthons using the first molecular reaction, thereby generating a second plurality of compounds;
viii) obtaining, for each respective instance of the first molecular reaction, a respective conversion value for the respective instance;
ix) selecting a second subset of instances from the plurality of instances based on at least the first threshold conversion value for the respective conversion value of each respective instance, wherein the automated synthesis platform informs the computing system of the subset of instances;
x) retraining the first model by using i) the second subset of instances as independent variables and ii) the corresponding conversion value of each instance of the second subset of instances as dependent variables, to guide adjustment of one or more parameters in the plurality of parameters of the first model, so that the first model produces a calculated conversion value in agreement with the corresponding conversion value of each instance of the second subset of instances upon input of the subset of instances into the first model; and
xi) using, subsequent to obtaining the updated plurality of parameters, the first model to search for and identify a reupdated plurality of normalized conditions for the first molecular reaction that collectively have an improved conversion value for the first molecular reaction relative to the updated plurality of normalized conditions.

15. The apparatus of claim 1, wherein the training v) is in accordance with a loss function, an assent function, or a regression.

16. The apparatus of claim 3, wherein the plurality of unlabeled molecules comprises 1000 or more unlabeled molecules or 10,000 or more unlabeled molecules.

17. The apparatus of claim 3, wherein the plurality of unlabeled molecules comprises 1×10⁶ or more unlabeled molecules.

18. The apparatus of claim 1, wherein each compound in the first plurality of compounds is an organic compound having a molecular weight of less than 500 Daltons, less than 1000 Daltons, less than 2000 Daltons, less than 4000 Daltons, less than 6000 Daltons, less than 8000 Daltons, less than 10000 Daltons, or less than 20000 Daltons.

19. An apparatus for automating synthesis of a compound using a first molecular reaction, wherein the first molecular reaction is a multistep synthesis, the apparatus comprising:
a) an automated synthesis platform; and
b) a computing system comprising one or more processors and memory addressable by the one or more processors, the memory storing a first model; wherein the automating comprises:
i) selecting the first molecular reaction;
ii) performing a first plurality of instances of the first molecular reaction using a first plurality of at least 4 synthons and a plurality of normalized conditions using the automated synthesis platform, comprising:
for each respective instance of the first molecular reaction, transforming, with the automated synthesis platform, at least a subset of the first plurality of synthons using the first molecular reaction, thereby generating a first plurality of compounds;
iii) obtaining, for each respective instance of the first molecular reaction, a respective conversion value for the respective instance;
iv) selecting a first subset of instances from the first plurality of instances based on at least a first threshold conversion value for the respective conversion value of each respective instance, wherein the automated synthesis platform informs the computing system of the first subset of instances; and
v) training the first model by using i) the first subset of instances as independent variables and ii) the corresponding conversion value of each instance of the first subset of instances as dependent variables, to guide adjustment of one or more parameters in a plurality of parameters of the first model, so that the first model produced conversion values are in agreement with the corresponding conversion value of each instance of the first subset of instances upon input of the first subset of instances into the first model.

20. An apparatus for selecting synthons for a molecular reaction, wherein the molecular reaction is a multistep molecular reaction, the apparatus comprising:
a) an automated synthesis platform; and
b) a computing system comprising one or more processors and memory addressable by the one or more processors; wherein the identifying comprises:
i) selecting the molecular reaction;
ii) performing a first plurality of instances of the molecular reaction using a plurality of at least 4 synthons and a plurality of normalized conditions using the automated synthesis platform, comprising:
for each respective instance of the molecular reaction, transforming, with the automated synthesis platform, at least a subset of the plurality of synthons using the molecular reaction, thereby generating a first plurality of compounds;
iii) obtaining, for each respective instance of the molecular reaction, a respective conversion value for the respective instance;
iv) selecting a first subset of instances from the plurality of instances based on at least a threshold conversion value for the respective conversion value of each respective instance; and
v) selecting a first subset of synthons that are enriched in the first subset of instances relative to the plurality of instances of the molecular reaction.
